(19) United States
(12) Patent Application Publication
(10) Pub. No.: US 2023/0330045 A1
(43) Pub. Date: Oct. 19, 2023

(54) THE ANTIVIRAL DRUG TILORONE IS A POTENT AND SELECTIVE INHIBITOR OF ACETYLCHOLINESTERASE

(71) Applicant: Collaborations Pharmaceuticals, Inc., Raleiigh, NC (US)

(72) Inventors: Patricia A. Vignaux, Raleigh, NC (US); Ana C. Puhl Rubio, Raleigh, NC (US); Sean Ekins, Raleigh, NC (US)

(21) Appl. No.: 18/026,779

(22) PCT Filed: Sep. 16, 2021
(86) PCT No.: PCT/US2021/050723

§ 371 Date: Mar. 16, 2023

Related U.S. Application Data
(60) Provisional application No. 63/079,376, filed on Sep. 16, 2020.

(51) Int. Cl. A61K 31/138; A61K 45/06

(52) U.S. Cl. CPC A61K 31/138; A61K 45/06

(57) ABSTRACT

The identification of new acetylcholinesterase (AChE) inhibitors is described. For example, tilorone was newly identified as an AChE inhibitor by use of a machine learning model followed by in vitro screening. The new AChE inhibitors can selectively inhibit AChE compared to butyrylcholinesterase (BuChE). Methods of inhibiting AChE and of treating or preventing diseases, disorders, and conditions treatable or preventable by AChE inhibition are also described. For example, methods of treating and/or preventing certain dermatological conditions and organophosphorous or nerve agent poisoning are described.

### RELATED APPLICATIONS

The presently disclosed subject matter claims the benefit of U.S. Provisional Patent Application Ser. No. 63/079,376 filed Sep. 16, 2020, the disclosure of which is incorporated herein by reference in its entirety.

### STATEMENT OF GOVERNMENT INTEREST

This invention was made with government support under Grant Numbers R44GM122196-02A1 and 3R44GM122196-0351 awarded by the National Institutes of Health. The government has certain rights in the invention.

### TECHNICAL FIELD

The presently disclosed subject matter relates to the identification of new inhibitors of acetylcholinesterase (AChE) and to the treatment or prevention of various diseases and conditions treatable or preventable by AChE inhibition, including organophosphorous (OP) poisoning and nerve agent poisoning. In some embodiments, the presently disclosed subject matter relates to the finding that tilorone is a selective and potent AChE inhibitor.

### ABBREVIATIONS

%=percent or percentage

° C.=degrees Celsius

A=angstrom

μL=microliter

μM=micromolar

2D=two dimensional

3D=three dimensional

AC=ASSAY CENTRAL®

AChE=acetylcholinesterase p AD=Alzheimer's Disease

Ada=AdaBoosted Decision Trees

Bnb=Naïve Bayesian

BuChE=butyrylcholinesterase

CNS=central nervous system

CWA=chemical warfare agent

DL=deep learning

DMSO=dimethyl sulfoxide

GABA=gamma-aminobutyric acid

H₂O₂=hydrogen peroxide

IC₅₀=50% inhibitory concentration

kcal=kilocalorie

knn=k-Nearest Neighbors

MIA=multivariate image analysis

mL=milliliter

mol=moles

MS=multiple sclerosis

nAChR=nicotinic acetylcholine receptor

nm=nanometer

nM=nanomolar

OD=optical density

OP=organophosphorous

PAS=peripheral anionic site

PC=positive control

PD=Parkinson's disease

PDB=protein data base

QSAR=quantitative structure activity relationship

rf=Random Forest

ROC=receiver operator characteristic

svc=Support Vector Classification

W=tryptophan

Y=tyrosine

### BACKGROUND

Acetylcholinesterase (AChE) is the enzyme responsible for terminating the majority of acetylcholine neurotransmission at neuromuscular junctions and cholinergic synapses. Unlike other neurotransmitters, acetylcholine is not taken up at the synapse to be recycled but is hydrolyzed by cholinesterases into choline and acetic acid. Because of the role of AChE in the central and peripheral nervous systems, inhibitors of this enzyme can be useful in mitigating the symptoms of some neurological disorders. For example, AChE inhibitors are prescribed for treating Alzheimer's Disease (AD), a progressive neurodegenerative disease, resulting in a concentrated loss of cholinergic neurons in the basal forebrain¹. As cholinergic neurons die, less acetylcholine is produced and AChE inhibitors like the FDA-approved donepezil, galantamine and rivastigmine, prevent depletion of this crucial neurotransmitter. These drugs are not curative, but instead are prescribed to slow the rate of cognitive decline caused by the disease. AChE inhibitors are also used in the treatment of other diseases, such as myasthenia gravis², Lewy Body dementia³, and glaucoma³⁴, and are currently under investigation for therapeutic efficacy in schizophrenia⁵ and Parkinson's disease dementia⁶. The AChE inhibitor physostigmine is also used as a therapy for acute cases of anticholinergic poisoning caused by anticholinergic agents like some antihistamines or tricyclic antidepressants.

Accordingly, there is an ongoing need for additional AChE inhibitors for use in treating or preventing these and other diseases, disorders and conditions. In particular, there is an ongoing need for additional AChE inhibitors that have improved potency and/or milder side-effects than those currently in use.

### SUMMARY

In some embodiments, the presently disclosed subject matter provides a method of inhibiting AChE in a sample comprising AChE, wherein the method comprises contacting the AChE in the sample with an effective amount of at least one compound selected from the group comprising tilorone, a tilorone analog, cetylpyridinium, bezedoxifene acetate, rifaximin, dequalinium chloride, agelasine, and pharmaceutically acceptable salts thereof. In some embodiments, the at least one compound is selected from tilorone, a tilorone analog and/or a pharmaceutically acceptable salt thereof, wherein said tilorone analog is a selective AChE inhibitor.

In some embodiments, the at least one compound has a 50% inhibitory concentration (IC₅₀) for human and/or eel AChE of about 100 nanomolar (nM) or less. In some embodiments, the at least one compound has an ICso for human AChE of about 75 nM or less and/or an IC₅₀ for eel AChE of about 15 nM or less. In some embodiments, the at least one compound has an ICso for human or eel AChE that is at least about 100 times lower than an IC₅₀ of the at least one compound for butyrylcholinesterase (BuChE), optionally wherein the at least one compound has an ICso for human or eel AChE that is at least about 1000 times lower than an IC₅₀ of the at least one compound for BuChE. In some embodiments, the tilorone or tilorone analog and/or pharmaceutically acceptable salt thereof exhibits pi-pi interactions with tryptophan 286 (W286) of the peripheral anionic site (PAS) of human AChE.

In some embodiments, the sample comprises a biological fluid, cell, cell extract, tissue, tissue extract, organ or whole organism.

In some embodiments, the tilorone or tilorone analog and/or pharmaceutically acceptable salt thereof has a structure of one of Formula (I) and Formula (II):

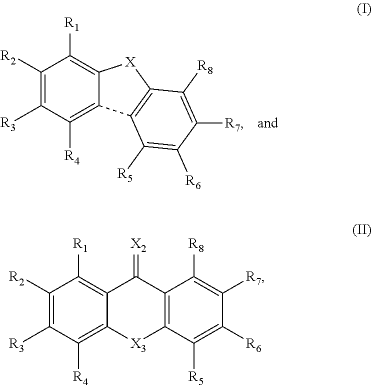

wherein: [symbol] is absent or a single bond; X is selected from the group comprising —C(═Z)—, —S(═O)₂, —CH₂, —O—, —S—, and —NH—; Z is selected from O, S, and CH₂; X₂ is selected from O, S, and CH₂; X₃ is selected from —C(═Z)—, —CH₂—, —O—, —S—, and —NH—; each of R₁, R₂, R₃, R₄, R₅, R₆, R₇, and R₈ is independently selected from the group comprising H, alkyl, amino, hydroxy, alkoxy, and —X4-L-N(R₉)₂; X₄ is selected from —O—, —S—, —NH—C(═O)—, —O—C(═O)—, —C(═O)—, —C(OH)—, and —C(═O)—O—; L is a bivalent linker moiety, optionally a C₁-C₆ alkylene group; and each R₉ is alkyl, optionally C₁-C₆ alkyl; aralkyl, or aryl, or wherein two R₉ together form a cyclic bivalent group; or a pharmaceutically acceptable salt thereof. In some embodiments, at least one of R₁-R₈ is —X4-L-N(₉)₂, optionally wherein one of R₁-R₄ is —X₄-L-N(R₉)₂ and one of R₅-R₈ is X₄-L-N(R₉)₂.

In some embodiments, the presently disclosed subject matter provides a method of treating or preventing a disease, disorder, or condition treatable or preventable by inhibition of AChE in a subject in need of treatment thereof and/or of extending the lifespan of a subject, wherein the disease, disorder or condition treatable or preventable by inhibition of AChE is selected from the group comprising a dermatological disorder, myasthenia gravis, glaucoma, multiple sclerosis, autoimmune encephalomyelitis, OP poisoning, nerve agent poisoning, and anticholinergic poisoning, wherein the method comprises administering to the subject an effective amount of at least one compound selected from the group comprising tilorone, a tilorone analog, cetylpyridinium, dequalinium chloride, bezedoxifene acetate, rifaximin, agelasine, and pharmaceutically acceptable salts thereof. In some embodiments, the method comprises administering to the subject an effective amount of tilorone or an analog and/or pharmaceutically acceptable salt thereof, wherein said tilorone analog is a selective inhibitor of AChE.

In some embodiments, the subject is a subject suffering from or suspected to be suffering from OP or nerve agent poisoning or is at risk of OP or nerve agent poisoning. In some embodiments, the subject is at risk for OP or nerve agent poisoning, and the subject is administered the at least one compound prior a potential exposure to an OP or a nerve agent. In some embodiments, the method further comprises administering to said subject one or more additional treatment agents for OP or nerve agent poisoning, optionally wherein said one or more additional treatment agents are selected from the group comprising atropine, pralidoxime or another oxime, a benzodiazepine, and physostigmine salicylate.

In some embodiments, the disease, disorder, or condition treatable or preventable by inhibition of AChE is a dermatological disorder selected from the group comprising a condition associated with Domodex brevis and/or Demodex folliculorum mites, a bacterial infection, acne, seborrheic dermatitis, perioral dermatitis, acneform rash, transient acantholytic dermatosis, acne necrotica milliaris, steroid induced dermatitis, primary irritation dermatitis, and rosacea.

In some embodiments, the at least one compound has an IC₅₀ for eel and/or human AChE of about 100 nM or less. In some embodiments, the at least one compound has an IC₅₀ for human AChE of about 75 nM or less and/or an IC₅₀ for eel AChE of about 15 nM or less.

In some embodiments, the at least one compound has an IC₅₀ for human or eel AChE that is at least about 100 times lower than an IC₅₀ of the at least one compound for BuChE, optionally wherein the at least one compound has an IC₅₀ for human or eel AChE that is at least about 1000 times lower than an IC₅₀ of the at least one compound for BuChE.

In some embodiments, the subject is a mammal, optionally a human. In some embodiments, the administering is performed via one of the group comprising oral administration, intravenous (IV) administration, intraperitoneal (IP) administration, topical administration, intracerebroventricular (ICV) administration, and intrathecal (IT) administration.

In some embodiments, the tilorone, tilorone analog and/or pharmaceutically acceptable salt thereof has a structure of one of Formula (I) and Formula (II):

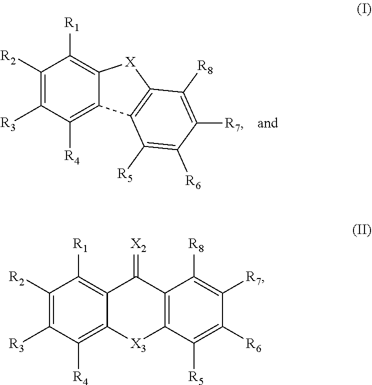

wherein: [symbol] is absent or a single bond; X is selected from the group comprising —C(═Z)—, —S(═O)₂—, —CH₂—, —O—, —S—, and —NH—; Z is selected from O, S, and CH₂; X₂ is selected from O, S, and CH₂; X₃ is selected from —C(═Z)—, —CH₂—, —O—, —S—, and —NH—; each of R₁, R₂, R₃, R₄, R₅, R₆, R₇, and R₈ is independently selected from the group comprising H, alkyl, amino, hydroxy,alkoxy, and —X₄-L-N(R₉)₂X₄ is selected from —O—, —S—, —NH—C(═O)—, —O—C(═O)—, —C(═O)—, —C(OH)—, and —C(═O)—O—; L is a bivalent linker moiety, optionally a C₁-C₆ alkylene group; and each R₉ is alkyl, optionally C₁-C₆ alkyl; aralkyl, or aryl, or wherein two R₉ together form a cyclic bivalent group; or a pharmaceutically acceptable salt thereof. In some embodiments, at least one of R1-R₈ is —X₄-L-N(R₉)₂, optionally wherein one of R₁-R₄ is —X₄-L-N(R₉)₂ and one of R₅-R₈ is X₄-L-N(R₉)₂.

In some embodiments, the method further comprises administering to the subject one or more additional therapeutic agents.

Accordingly, it is an object of the presently disclosed subject matter to provide methods of inhibiting AChE and treating or preventing diseases, disorders, and conditions treatable or preventable by AChE inhibition in a subject in need thereof and/or extending the lifespan of a subject.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIGS. 1A-1C. Machine learning model five-fold receiver operating characteristic (ROC) curves for (FIG. 1A) eel acetylcholinesterase (AChE); (FIG. 1B) human AChE; and (FIG. 1C) human butyrylcholinesterase (BuChE).

FIG. 2 is a series of radar plots showing the comparison of different machine learning algorithms for models built for, from left to right, eel acethylcholinesterase (AChE), human AChE, and human butyrylcholinesterase (BuChE). Radar plots depict the metrics resulting from five-fold cross-validation. AC=ASSAY CENTRAL® (Bayesian), rf=Random Forest, knn=k-Nearest Neighbors, svc=Support Vector Classification, bnb=Naïve Bayesian, ada=AdaBoosted Decision Trees, DL=Deep Learning.

FIG. 3 is a series of graphs screening for inhibitors of eel acetylcholinesterase (AChE) that reveals novel inhibitors. 2421 compounds from a compound screening library were screened for activity in vitro. Activity was defined as greater than or equal to 50 percent (≥50%) inhibition of enzyme activity compared to dimethyl sulfoxide (DMSO) control. The compounds marked with shaded circles were known inhibitors of AChE, while the compounds marked in unshaded circles, including cetylpyridinium (Plate 1), bezedoxifene acetate (Plate 2), rifaximin (Plate 3), tilorone (Plate 4), dequalinium chloride (Plate 5) and agelasine (Plate 7), were not previously described as inhibitors.

FIGS. 4A and 4B. Assay performance for the eel acetylcholinesterase (AChE) screen. FIG. 4A is a graph showing signal to background ratio for each plate. Ratio represents average signal enzyme+substrate/average signal substrate. Average signal/background=8.49±1.10. FIG. 4B is a graph showing Z′ for each plate. Average Z′=0.91±0.03.

FIGS. 5A-5F. Series of graphs from a test for compound interference in the eel acetylcholinesterase (AChE) assay. Six compounds (FIG. 5A, agelasine; FIG. 5B, dequalinium chloride; FIG. 5C, cetylpyridinium; FIG. 5D, rifaximin; FIG. 5E, bezedoxifene acetate; and FIG. 5F, tilorone) scored as novel inhibitors in the AChE screen (data shown in black circles). Parallel dose-response reactions were run to determine the ability of the compound to disrupt assay signal generation in an acetylcholinesterase reaction versus a reaction containing the reaction product hydrogen peroxide (H₂O₂) (data shown in grey circles) The percent (%) activity of the enzymatic reactions are based upon a positive control of eel AChE and 1% dimethyl sulfoxide (DMSO). The percent activity of the H₂O₂ reactions are based upon the maximum signal of reaction with 5 micromolar (μM) H₂O₂ and 1% DMSO.

FIGS. 6A-6C. Dose-response of three novel inhibitors for eel acetylcholinesterase (eel AChE). Sixteen-point serial dilutions beginning at 100 NM were performed for agelasine (FIG. 6A; n=2), dequalinium chloride (FIG. 6B, n=4), and tilorone (FIG. 6C, n=4). Reactions were normalized to enzyme with 1 percent (%) dimethyl sulfoxide (DMSO) (0% inhibition) and buffer reaction with no enzyme (100% inhibition). Fifty percent inhibitory concentration (IC₅₀) values were generated using a non-linear regression log(inhibitor) vs. response equation with four parameters. The Hill slope for each compound are: agelasine=0.79, dequalinium chloride=0.87, tilorone=0.62.

FIGS. 7A-7C. Selective inhibition of human acetylcholinesterase (hAChE) by tilorone. FIG. 7A is a graph of dose-response curves of known and novel inhibitors of hAChE, using a colorimetric assay. Tilorone data is shown in circles and donepezil data in squares. A two-fold, 16-point serial dilution was tested for each compound (n=2), beginning at 20 micromolar (μM). Fifty percent inhibitory concentrations (IC₅₀) curves were generated using non-linear regression log(inhibitor) vs. response equation with four parameters with constraints at the top (100) and bottom (0) of the curves. The Hill slopes for each curve are tilorone=0.84 and donepezil=0.74. FIG. 7B is a graph showing secondary in vitro inhibition analysis of tilorone. A two-fold serial dilution of tilorone beginning at 5 μM was performed and tested for inhibition using photometric analysis. Results were given as

Inhibition of Control Values (n=2), and curves was re-created using the following supplied values: IC₅₀=56 nM, Top=99, Bottom=−7, Hill slope=1.0. Error bars representing standard deviation are too small be shown on graph. FIG. 7C is a graph showing single-point determination of the inhibitory effect of 100 μM tilorone on human butyrylcholinesterase (BuChE) compared to known inhibitors physostigmine and rivastigmine tartrate (n=2). The percent activity of the enzymatic reactions are based upon a positive control of BuChE and 1% dimethyl sulfoxide (DMSO).

FIG. 8 is a graph of the absorbance at 412 nanometers (nm) of 150 micromolar (μM) of the 5-thio-2-nitro-benzoic acid standard in the presence of dimethyl sulfoxide (DMSO) or 100 μM compound (n=2). Unpaired t-test 8.4.3, P=0.2536.

FIGS. 9A and 9B are receiver operator characteristic (ROC) plots for external testing with experimental data from this study. FIG. 9A is for eel acetylcholinesterase (AChE). FIG. 9B is for human AChE.

FIGS. 10A-10E show results of docking studies of tilorone into human acetylcholinesterase (AChE). FIG. 10A is a schematic drawing of a crystal structure of donepezil with recombinant human AChE (rhAChE; PDB 4EY7) used to dock tilorone. FIG. 10B is a schematic drawing of donepezil AChE docking shown with residues deemed important for protein-ligand interactions. FIG. 10C is a schematic drawing showing docking of tilorone in AChE, shown with the same residues as FIG. 10B. FIG. 10D is a schematic drawing of donepezil in the three-dimensional (3D) space of the AChE gorge. FIG. 10E is a schematic drawing of tilorone in the 3D space of the AChE gorge.

FIGS. 11A and 11B. Contrasting pi-pi interactions between the aromatic residues in acetylcholinesterase (AChE) and two inhibitors. FIG. 11A is a schematic drawing of a crystal structure of donepezil (PDB 4EY7) displaying pi interactions between its ring structures and the residues tryptophan 86 (W86) and tryptophan 286 (W286) of AChE. FIG. 11B is a schematic drawing of the AChE docking of tilorone, suggesting pi interactions between its core ring structure and tyrosine 341 (Y341) and W286.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, capacitance, specific capacity, discharge capacity, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

A disease or disorder is “alleviated” if the severity of a symptom of the disease, condition, or disorder, or the frequency at which such a symptom is experienced by a subject, or both, are reduced.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As use herein, the terms “administration of” and/or “administering” a compound should be understood to refer to providing a compound of the presently disclosed subject matter to a subject in need of treatment.

As used herein, an “agonist” is a composition of matter which, when administered to a mammal such as a human, enhances or extends a biological activity attributable to the level or presence of a target compound or molecule of interest in the subject.

As used herein, amino acids are represented by the full name thereof, by the three letter code corresponding thereto, or by the one-letter code corresponding thereto, as indicated in Table A, below:

TABLE A
Amino Acid Codes and Functionally Equivalent Codons

| | 3-Letter | 1-Letter | Functionally Equivalent |
|---|---|---|---|
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

Amino acids have the following general structure:

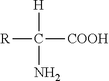

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

The term “biological sample”, as used herein, refers to samples obtained from a subject, including but not limited to skin, hair, tissue, blood, plasma, cells, sweat, and urine.

“Co-administer” can include simultaneous and/or sequential administration of two or more agents.

A “compound,” as used herein, refers to any type of substance or agent that is can be considered a drug, or a candidate for use as a drug, as well as combinations and mixtures of the above.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

As used herein, a “derivative” of a compound refers to a chemical compound that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and “administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample can of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in an animal (for example, without limitation, a human or other mammal). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

The term “prevent”, as used herein, means to stop something from happening, or taking advance measures against something possible or probable from happening. In the context of medicine, “prevention” generally refers to action taken to decrease the chance of getting a disease or condition or a symptom thereof. It is noted that “prevention” need not be absolute, and thus can occur as a matter of degree. For example, prevention can involve preventing some, but not all, symptoms or undesirable biological effects associated with a particular disease or disorder.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of the presently disclosed subject matter.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing or eliminating, that results in the improvement of the condition, disease, disorder, and the like, or ameliorating a symptom thereof.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

As used herein the term “alkyl” can refer to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc) in the backbone of a ring structure. Nitrogen-containing heteroaryl moieties include, but are not limited to, pyridine, imidazole, benzimidazole, pyrazole, pyrazine, triazine, pyrimidine, and the like.

“Cyclic” and “Cycloalkyl” refer to a non-aromatic mono- or multicyclic ring system of about 3 to about 10 carbon atoms. The cycloalkyl group can be optionally partially unsaturated. The cycloalkyl group can be also optionally substituted with an alkyl group substituent as defined herein, oxo and/or alkylene. There can be optionally inserted along the cyclic alkyl chain one or more oxygen, sulphur or substituted or unsubstituted nitrogen atoms, wherein the nitrogen substituent is hydrogen, lower alkyl, or aryl, thus providing a heterocyclic group. Representative monocyclic cycloalkyl rings include cyclopropyl, cyclobutyl, cyclopentyl, cyclohexyl and cycloheptyl. Exemplary multicyclic cycloalkyl rings include adamantyl, octahydronaphthyl, decalin, camphor, camphane, and noradamantyl.

The term “heterocyclic” refers to a non-aromatic or aromatic mono- or multicyclic ring system of about 3 to about 12 atoms that comprises at least one heteroatom, e.g., N, O, or S. The group can be saturated, partially unsaturated, or unsaturated. Exemplary heterocyclic groups include, but are not limited to, furanyl, pyrrolyl, pyridinyl, pyranyl, piperidinyl, morpholinyl, dioxanyl, pyrrolidinyl, oxanyl, thiolanyl, and thiophenyl. Heterocyclic groups can be unsubstituted or substituted with one or more alkyl group substituents or aryl group substituents.

“Aralkyl” refers to an -alkyl-aryl group, optionally wherein the alkyl and/or aryl moiety is substituted. An exemplary aralkyl group is benzyl, i.e., —CH₂C₆H₅.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)₁—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “arylene” refers to a bivalent aromatic group, e.g., a bivalent phenyl or napthyl group. The arylene group can optionally be substituted with one or more aryl group substituents and/or include one or more heteroatoms.

The term “aralkylene” refers to a bivalent group that includes both aromatic and non-aromatic groups.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamino” and “aminoaryl” refer to the group —N(R)₂ wherein each R is H, aryl, or substituted aryl, and wherein at least one R is aryl or substituted aryl, e.g., aniline (i.e., —NHC₆H₅).

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “halo”, “halide”, or “halogen” as used herein refer to fluoro, chloro, bromo, and iodo groups.

The terms “hydroxyl” and “hydroxy” refer to the —OH group.

The terms “mercapto” or “thiol” refer to the —SH group.

The terms “carboxylate” and “carboxylic acid” can refer to the groups —C(═O)O⁻ and —C(═O)OH, respectively. The term “carboxyl” can also refer to the —C(═O)OH group. In some embodiments, “carboxylate” or “carboxyl” can refer to either the —C(═O)O⁻ or —C(═O)OH group.

The term “carbonyl” refers to the —C(═O)— group.

The term “carbamate refers to the group —O—C(═O)—NH— or —O—C(═O)—NR′—, wherein R′ is alkyl, substituted alkyl, aralkyl, substituted aralkyl, aryl, or substituted aryl.

By the term “protecting group” is meant a group which inhibits or suppresses undesirable chemical reactions, but which is designed to be sufficiently reactive that it can be cleaved from the functional group in question to obtain the desired product under mild enough conditions that do not modify the rest of the molecule. Protecting groups are well known to those skilled in the art and are described in ‘Protective Groups in Organic Synthesis’, Theorodora W. Greene and Peter G. M. Wuts, (Third Edition, John Wiley & Sons, 1999).

The term “hydrophilic” can refer to a compound or chemical species or functional group that dissolves or preferentially dissolves in water and/or aqueous solutions.

The term “hydrophobic” refers to compounds, chemical species or functional groups, that do not significantly dissolve in water and/or aqueous solutions and/or which preferentially dissolve in fats and/or non-aqueous solutions.

A dashed line representing a bond in a chemical formula indicates that the bond can be either present or absent. For example, the chemical structure:

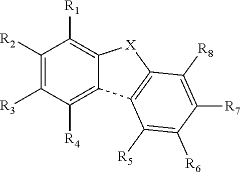

refers to compounds where the two outer six-membered ring can be joined by a direct bond and the —X— linkage or only by the —X— linkage.

The term “monovalent” as used herein refers to a chemical moiety that has one site available for chemical bonding to another chemical moiety. Thus, a “monovalent moiety” can be a part of whole molecule that is attached to the remainder of the whole molecule via an attachment at one site on the monovalent moiety.

The term “bivalent” as used herein refers to a chemical moiety that has two sites available for chemical bonding to another chemical moiety or moieties.

### II. General Considerations

AChE is a drug target of interest in neurological disorders, such as Alzheimer's Disease, Lewy Body dementia, and Parkinson's Disease dementia, as well as for other conditions, such as, but not limited to myasthenia gravis and anticholinergic poisoning. Thus, there has been much interest in finding new AChE inhibitors in recent years. One strategy is to look for novel inhibitors from natural sources. For example, galantamine was isolated from the snowdrop Galanthus nivalis and Huperzine A was isolated from the club moss Huperzia serrata⁸, and AChE inhibition has continued to be found in the direct extracts of plants^{9,10} and marine sponges.^{11,12} Moreover, synthetic analogs and derivatives of these natural products have been synthesized in an effort to find more AChE inhibitors, and derivatives from active compounds in peppers¹³, and trees^{14,15}, as well as derivatives of whole chemical classes of natural compounds like diterpenoids¹⁶, flavonoids^{17,18} and coumarins^{19,20} have been produced. Additionally, compounds either derived from or incorporating the commercial inhibitors donepezi^{21,22}, galantamine²³, or tacrine²⁴⁻²⁷ have been developed. Other approaches have focused on the shape of the enzyme itself and specifically designed molecules to interact with key parts of the AChE enzyme, like the peripheral anionic site (PAS).^{28,29}

In recent years, computational approaches have been used to identify AChE inhibitors in silico. These have included structure-based pharmacophore modeling³⁰, machine learning³¹, molecular docking³²⁻³⁴, 2D and 3D similarity searches³⁵, MIA-QSAR modeling³⁶, or combinations of these strategies.³⁷⁻³⁹

According to one aspect of the presently disclosed subject matter, the identification of new AChE inhibitors was accomplished by a combination of a machine learning approach using literature data and high throughput screening. In some embodiments, a docking approach was also used to visualize the interactions of AChE with the newly identified inhibitors. For instance, Bayesian machine learning models were generated with literature data for eel and human AChE inhibitors and butyrylcholinesterase (BuChE) inhibitors from ChEMBL, the chemical database of bioactive molecules with drug-like properties maintained by the European Bioinformatics Institute (EBI) of the European Molecular Biology Laboratory (EMBL), and compared with other machine learning methods. High throughput screens were performed to generate test sets for the eel and human AChE inhibitor models, which newly identified several molecules, including agelasine, dequalinium chloride, cetylpyridinium, rifaximin, tilorone, and bezedoxifene acetate, as AChE inhibitors. For example, tilorone, is an antiviral drug in use outside the United States and has recently been investigated for anti-ebolavirus activity in vitro and in vivo. According to the presently disclosed subject matter, tilorone was identified as a sub-micromolar AChE inhibitor. More particularly, as described hereinbelow, tilorone displayed potent AChE inhibition for both the eel (IC₅₀=14.4 nM) and human (IC₅₀=64.4 nM) AChE enzymes, but not the closely related BuChE (IC₅₀>50 NM). Based on docking studies, and without being bound to any one theory, it appears that this selectivity is likely due to an interaction with a hydrophobic residue (W286) in the peripheral anionic site (PAS) of AChE that is absent in BuChE. A pharmacological safety profile also demonstrated that tilorone (at a 1 μM concentration) only appreciably inhibited AChE out of 44 toxicology target proteins. Taken together, the data suggests a role for the newly identified AChE inhibitors and their analogs, e.g., tilorone and its analogs, in therapeutic applications that can benefit from AChE inhibition (e.g., non-covalent AChE inhibition), including, but not limited to, treatment and/or prevention of neurological disorders, cognitive disorders, dermatological conditions, organophosphorous (OP) poisoning, nerve agent poisoning, myasthenia gravis, glaucoma, multiple sclerosis (MS), autoimmune encephalomyelitis, and anticholinergic poisoning, as well as in use for extending lifespan.

In particular, there is an ongoing need for new agents for the treatment of OP and/or nerve agent poisoning. The use of organophosphate compounds in war and as pesticides has resulted in a rising number of cases of acute and delayed intoxication over the past 40 years, resulting in damage to the peripheral and central nervous systems, myopathy, psychosis, general paralysis, and death.

OPs are thought to affect the activity of AChE in a two-stage process. First, the OP compound can form a covalent adduct with a catalytic serine of the enzyme, thereby immediately inhibiting the function of AChE. Once deactivated, the AChE enzyme then undergoes a dealkylation reaction known as “aging.” This dealkylation reaction is considered irreversible, thereby rendering the “aged” AChE as non-functional. These actions result in the prevention of the breakdown of acetylcholine, resulting in a buildup which can lead to hyperactivity of the nervous system. Acetylcholine is not destroyed and continues to stimulate the muscarinic receptor sites (exocrine glands and smooth muscles) and the nicotinic receptor sites (skeletal muscles).

Exposure to OPs can cause symptoms ranging from mild (twitching, trembling) to severe (paralyzed breathing, convulsions), and in extreme cases, death, depending on the type and amount of substance involved. The action of organophosphates and carbamates makes them very effective as pesticides for controlling insects and other pests. OP pesticides (e.g., insecticides and herbicides) include, but are not limited to, chlorpyrifos, parathion, malathion, diazinon, fenthion, diclorvos, tribufus, and the like. Unfortunately, when humans or other mammals breathe or are otherwise exposed to these compounds, they are subjected to the same negative effects.

The devastating impact of certain OP pesticides on humans has led to the development of similar compounds as “nerve gases” or “nerve agents” as chemical warfare agents (CWAs) or for use in terrorist attacks. These compounds are related to organophosphorus insecticides, in that they are both esters of phosphoric acid. Nerve agents include, but are not limited to, diisopropylfluorophosphate (DFP), GA (tabun), GB (sarin), GD (soman), CF (cyclosarin), GE, CV, yE, VG (amiton), VM, VR (RVX or Russian VX), VS, and VX. Many nerve agents can be classified into the C-series or V-series based upon their physical properties and toxicities. C-series nerve agents are volatile liquids at room temperature and can be employed in liquid or vapor form. V series nerve agents, such as VX, are persistent liquid substances which can remain on material, equipment, and terrain for long periods. V-series nerve agents are generally more toxic than C-series nerve agents. Under temperate conditions, many nerve agents are clear colorless liquids, which are difficult to detect. A more recently developed class of nerve agents, Novichoks, are binary agents that can be dispersed as a fine powder, rather than as a gas or vapor.

Current treatment of organophosphate poisoning includes post-exposure intravenous or intramuscular administration of various combinations of drugs, including carbamates (e.g., pyridostigmine), anti-muscarinics (e.g., atropine), and ChE-reactivators such pralidoxime chloride (2-PAM, Protopam). Benzodiazepine anticonvulsives (e.g., diazepam) can also be administered. However, there is still a need for additional approaches to addressing threats like OP poisoning and nerve agent poisoning.

There is also precedent for drugs approved for one application being repurposed as potential countermeasures. The FDA approved drug galantamine, which is both a weak competitive inhibitor of AChE and a positive allosteric modulator (PAM) of the α7-nicotinic acetylcholine receptor (α7-nAChR)⁹¹ when given as a pretreatment has been shown to improve survival following exposure with several nerve agents.^{92,93} In particular, galantamine has also recently demonstrated in vivo efficacy in guinea pig as a pretreatment or post treatment⁹² and as an oral pretreatment in non-human primates⁹⁴ against soman exposure. Accordingly, the presently disclosed AChE inhibitory activity of agelasine, dequalinium chloride, cetylpyridinium, rifaximin, tilorone, and bezedoxifene acetate indicates that these compounds can also find use as countermeasures for OP and nerve agent poisoning.

### III. Methods of Inhibiting AChE

According to some aspects, the presently disclosed subject matter provides a method of inhibiting AChE in a sample comprising AChE, wherein the method comprises contacting the AChE in the sample with an effective amount of at least one compound selected from the group comprising tilorone, a tilorone analog, cetylpyridinium, bezedoxifene acetate, rifaximin, dequalinium chloride, agelasine, and pharmaceutically acceptable salts thereof. In some embodiments, the at least one compound is selected from tilorone, a tilorone analog and/or a pharmaceutically acceptable salt thereof. In some embodiments, the at least one compound (e.g., the tilorone analog and/or pharmaceutically acceptable salt thereof) is a selective AChE inhibitor. Thus, the presently disclosed subject matter provides, in some aspects, new identified AChE inhibitors and their analogs, including tilorone, a tilorone analog, cetylpyridinium, bezedoxifene acetate, rifaximin, dequalinium chloride, agelasine, and pharmaceutically acceptable salts thereof, for use in inhibiting AChE.

For example, in some embodiments, the at least one compound has a 50% inhibitory concentration (IC₅₀) for AChE (e.g., human and/or eel AChE) of about 100 nanomolar (nM) or less (e.g., about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 15 nM or less, or about 10 nM or less). In some embodiments, the at least one compound has an IC₅₀ for AChE (e.g., human and/or eel AChE) between about 1 nM and about 100 nM (e.g., about 1, 5, 10, 15, 20, 25, 30, 35, 40, 45, 50, 55, 60, 65, 70, 75, 80, 85, 90, 95, or about 100 nM). In some embodiments, the at least one compound has an IC₅₀ for human AChE of about 75 nM or less. In some embodiments, the at least one compound has an IC₅₀ for human AChE of about 50 nM to about 75 nM. Alternatively or additionally, in some embodiments, the at least one compound has an IC₅₀ for eel AChE of about 15 nM or less (e.g., between about 1 nM and about 15 nM).

In some embodiments, the at least one compound (e.g., the tilorone, tilorone analog and/or pharmaceutically acceptable salt thereof) selectively inhibits AChE (e.g., human and/or eel AChE) compared to other cholinesterases. In some embodiments, the at least one compound selectively inhibits AChE (e.g., human or eel AChE) compared to butyrylcholinesterase (BuChE) (e.g., human and/or eel BuChE). In some embodiments, the at least one compound has an IC₅₀ for AChE (e.g., human or eel AChE) that is at least about 100 times lower than that compound's IC₅₀ for BuChE (e.g., human or eel BuChE). In some embodiments, the at least one compound's IC₅₀ for AChE (e.g., human or eel AChE) is at least about 200, 300, 400, 500, 600, 700, 800, or 900 times lower than its IC₅₀ for BuChE (e.g., human or eel BuChE). In some embodiments, the at least one compound has an IC₅₀ for AChE (e.g., human or eel AChE) that is at least about 1000 times lower than its IC₅₀ for BuChE (e.g., human or eel BuChE). In some embodiments, the at least one compound has an IC₅₀ for AChE (e.g., human or eel AChE) that is about 1000, about 1100, about 1200, or about 1300 times lower than its IC₅₀ for BuChE (e.g., human or eel BuChE).

In some embodiments, the at least one compound (e.g., the tilorone or tilorone analog and/or pharmaceutically acceptable salt thereof) binds in the active site gorge of the AChE. In some embodiments, the at least one compound (e.g., the tilorone or tilorone analog and/or pharmaceutically acceptable salt thereof) exhibits pi-pi interactions with W286 of the peripheral anionic site (PAS) of human AChE (hAChE). In some embodiments, the at least one compound (e.g., the tilorone or tilorone analog and/or pharmaceutically acceptable salt thereof) exhibits pi-pi interactions with Y341 of hAChE. In some embodiments, the at least one compound is also an agonist of the alpha7 nicotinic acetylcholine receptor (α7 nAChR).

The sample comprising AChE can be any suitable sample, including aqueous samples comprising AChE. In some embodiments, the sample comprises a mammalian AChE. In some embodiments, the sample comprises human AChE. In some embodiments, the sample comprises one or more additional components, such as, but not limited to, a buffer or other pH adjusting agent, one or more additional enzymes or biological receptors, one or more other cholinesterase inhibitors, activators, or substrates. In some embodiments, the sample comprises a biological fluid (e.g., saliva, blood, plasma, etc.), a cell, a cell extract, a tissue, a tissue extract, an organ, or whole organism (e.g., a living organism, such as a mammal).

In some embodiments, the tilorone analog for use according to the presently disclosed subject matter is an analog that selectively inhibits AChE (e.g., compared to other cholinesterases, such as BuChE). In some embodiments, the tilorone analog has an IC₅₀ for an AChE (e.g., human or eel AChE) that is at least about 100 times, about 200 times, about 300 times, about 400 times, about 500 times, about 600 times, about 700 times, about 800 times, about 900 times, about 1000 times, about 1100 times, about 1200 times, or about 1300 times lower than its IC₅₀ for BuChE (e.g., human or eel BuChE). Suitable tilorone analogs of the presently disclosed subject matter generally include compounds that comprise a central scaffold comprising two or more aromatic rings that are coplanar and where the central scaffold has a similar length (e.g., ±about 25%, ±about 20%, ±about 15%, ±about 10%, or ±about 5%) to that of the fluoren-9-one central scaffold of tilorone. In some embodiments, the tilorone analog comprises a dicyclic or tricyclic central scaffold. In some embodiments, the central scaffold is selected from the group including, but not limited to, fluorene, fluoren-9one, 9-methylene fluorene (i.e., dibenzofulvene), dibenzofuran, dibenzothiophene, dibenzothiophene sulfone, 9,9-anthroquinone, carbazole, anthrone, and benzophenone. Typically, the tilorone analog further includes one or more aryl group substituents. In some embodiments, the tilorone analog includes at least two aryl group substituents, which can be the same or different, wherein at least one of the at least two aryl group substituents is attached to one aromatic ring of the central scaffold (e.g., one of the outer aromatic rings of the central scaffold) and another one of the at least two aryl group substituents is attached to another aromatic ring (e.g., the other outer aromatic ring of the central scaffold). In some embodiments, at least one aryl group substituent comprises a tertiary amine. In some embodiments, at least one aryl group substituent can be involved in hydrogen bonding interactions (e.g., a hydroxyl group).

In some embodiments, the tilorone or tilorone analog and/or pharmaceutically acceptable salt thereof has a structure of one of Formula (I) and Formula (II):

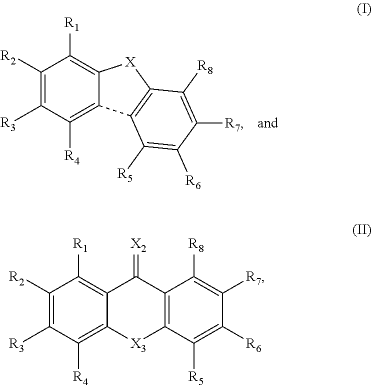

wherein [symbol] is absent or a single bond; X is selected from the group comprising —C(═Z)—, —S(═O)₂—, —CH₂—, —O—, —S—, and —NH—; Z is selected from O, S, and CH₂; X₂ is selected from O, S, and CH₂; X₃ is selected from —C(═Z)—, —CH₂—, —O—, —S—, and —NH—; each of R₁, R₂, R₃, R₄, R₅, R₆, R₇, and R₈ is independently selected from the group comprising H, alkyl (e.g., C₁-C₆ alkyl), amino, hydroxy, alkoxy (e.g., C₁-C₆ alkoxy), and —X₄-L-N(R₉)₂; X₄ is selected from —O—, —S—, —NH—C(═O)—, —O—C(═O)—, —C(═O)—, —C(OH)—and —C(═O)—O—; L is a bivalent linker moiety (e.g., an alkylene, arylene, or aralkylene group), optionally a C₁-C₆ alkylene group; and each R₉ is alkyl, optionally C₁-C₈ or C₁-C₆ alkyl; aralkyl, or aryl, or wherein two R₉ together form a cyclic bivalent group (e.g., so that the two R₉ groups together with the nitrogen atom to which they are attached form a ring, such as a pyrrolidine, pyrrole, pyridine, piperidine, morpholine, or diazinane group); or a pharmaceutically acceptable salt thereof.

In some embodiments, at least one of R₁-R₈ comprises a tertiary amino group, e.g., is a group of the formula —X₄-L-N(R₉)₂. In some embodiments, at least one of R₂, R₃, R₆, and R₇ comprises a tertiary amino group, e.g., is a group of the formula —X₄-L-N(R₉)₂. In some embodiments, one of R₁-R₄ comprises a tertiary amino group (e.g., is a group of the formula X₄-L-N(R₉)₂) and one of R₅-R₈ comprises a tertiary amino group (e.g., is a group of the formula X₄-L-N(R₉)₂). In some embodiments, one of R₂ and R₃ comprises a tertiary amino group (e.g., is a group of the formula —X₄-L-N(R₉)₂) and one of R₆ and R₇ comprises a tertiary amino group (e.g., is a group of the formula X₄-L-N(R₉)₂). In some embodiments, X₄ is selected from —O— and —C(═O)—. In some embodiments, L is C₁-C₆ alkylene (e.g., methylene, ethylene, propylene, butylene, pentylene, hexylene). In some embodiments, each R₉ is alkyl. In some embodiments, each R₉ is methyl or ethyl.

In some embodiments, one or two of R₁-R₈ is hydroxy. For example, in some embodiments, the tilorone analog is 2,7-dihydroxyfluoren-9-one.

In some embodiments, the tilorone analog is a compound of Formula (II). In some embodiments, X₃ is —CH₂— or —C(═O)—. In some embodiments, X₂ is 0.

In some embodiments, the tilorone analog is a compound of Formula (I). In some embodiments, [symbol] is absent and X is —C(═Z)—(e.g., —C(═O)—). In some embodiments, [symbol] is present (e.g., is a single bond) and X is selected from —C(═Z)—, —S(═O)₂—, —CH₂—, —O—, —S—, and —NH—. In some embodiments, X is selected from —C(═O)—, —C(═CH₂)—, —CH₂—, and —O—.

Tilorone analogs of the presently disclosed subject matter can be prepared using methods and organic group transformations known in the art. Various methods of preparing tilorone analogs (including 1,5-disubstituted, 2,5-disubstituted, and 3,5-disubstituted tilorone analogs) have been previously described, for example, in U.S. Pat. No. 6,004,959, the disclosure of which is incorporated herein by reference in its entirety. For example, the analogs can be prepared from phenyl-substituted oxazoline compounds where the phenyl group is itself substituted by one or more alkoxy group. The phenyl-substituted oxazoline is then reacted with a phenyl Grignard reagent (e.g., where the phenyl Grignard reagent is also alkoxy-substituted) to provide a diphenyl oxazoline. The diphenyl oxazoline is then converted to a diphenyl acid and cyclized (e.g., using thionyl chloride and SnCl₄) or can be cyclized after conversion of the diphenyl oxazoline to a carboxamide intermediate.

Synthetic methods for preparing tilorone analogs are also described, for example, in Jones et al. (J. Org. Chem., 42(25):4144-4146 (1977)); Jones et al. (J. Org. Chem., 61(11):3920-3922 (1996)); Alberecht et al. (J. Med. Chem., 17(8):886-889 (1974)); Andrews et al. (J. Med. Chem, 17(8):882-886 (1974)); and Zhang et al. (Molecules, 20(12):21458-21463 (2015)). For example, as described in Alberecht et al. (J. Med. Chem., 17(8):886-889 (1974)), di-basic-substituted fluorenes can be prepared by Friedel—Crafts acylation of fluorene using a ω-chloroalkanoyl chloride followed by amination in the presences of excess amine (e.g., a dialkylamine) and, if desired, oxidized (e.g., using molecular oxygen and benzyltrimethylammonium hydroxide (Triton B) in pyridine) to provide the corresponding fluoren-9-one. The corresponding dimethanols of the fluorenes can be prepared via sodium borohydride reduction.

As described in Jones et al. (J. Org. Chem., 42(25):4144-4146 (1977)) asymmetric 2,7-disubstituted fluorenes can be prepared starting with fluorene. See Scheme 1, below. Friedel—Crafts acylation of fluorene A with acetyl chloride or acetyl anhydride in the presence of a Lewis acid catalyst, such as aluminum chloride, provides a mono-acylated fluorene intermediate B. Reaction of intermediate B with oxalyl chloride provides carboxylic acid intermediate C, which can then be esterified with an alcohol (e.g., ethanol) using acid catalysis, providing ester intermediate D. Ester D can be reacted with a dialkylamine salt to provide a mono-cationic disubstituted fluorene E. Alternatively, intermediate B can undergo Baeyer-Villiger oxidation (in the presence of a peracid, such as meta-chloroperbenzoic acid (MCPBA) or hydrogen peroxide) to provide hydroxy-substituted intermediate F. Alkylation of F with a halo-substituted amine can provide the fluorene ether G. Reaction of G with a halo-substituted acid chloride can provide di-substituted intermediate H, which can then be reacted with an amine to provide the di-cationic fluorene I. If desired, the fluorene compounds can be oxidized to provide the corresponding fluoren-9-one tilorone analog via oxidation of the fluorene (e.g., using a crown ether and KOH or other conditions known in the art, such as using molecular oxygen with Triton B in pyridine).

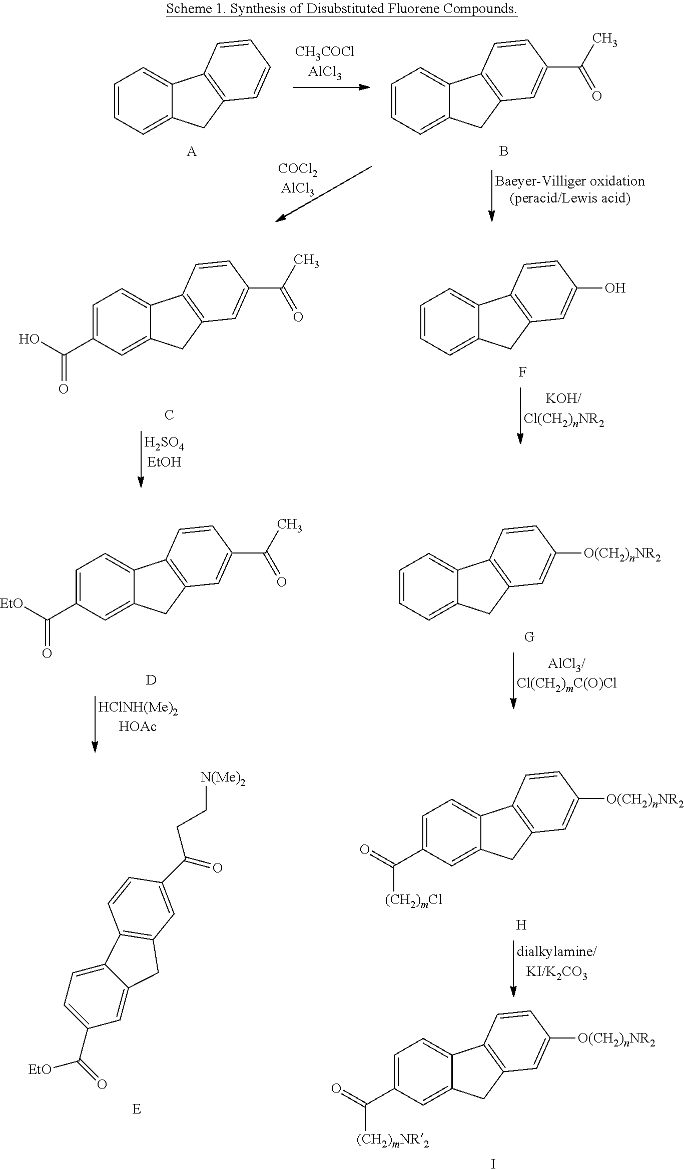

Zhang et al. (Molecules, 20(12): 21458-21463 (2015) describes the synthesis of 2,7-dihydroxyfluorene-9-one and the preparation of symmetric di-basic tilorone analogs via bis etherification with a dialkylaminoalkylene halide (e.g., 2-diethylaminoethylchloride in the case of tilorone itself).

Tilorone analogs with other scaffolds can be prepared via analogous methods. For example, dibenzofuran, carbazole, 9-methylene fluorene, and dibenzothiophene analogs can be prepared by etherification of hydroxy-substituted starting materials (e.g., 2,7-dihydroxy-dibenzofuran) with halo-substituted dialkylaminoalkanes or via Friedel-Crafts acylation reactions of the unsubstituted scaffold using ω-chloroalkanoyl chlorides followed by amination in the presences of excess amine. As needed, the nitrogen atom of the carbazole scaffold intermediate can be protected by a nitrogen protecting group as known in the art. Dibenzothiophene sulfones can be prepared via oxidation of the sulfur of the corresponding dibenzothiophenes.

### IV. Methods of Treating Diseases, Disorders, and Conditions Treatable or Preventable by AChE Inhibition

As described hereinabove, AChE is a target of interest for the treatment of neurological diseases and disorders. It is also a target of interest in a variety of other diseases, disorders, and conditions, such as, for example, certain dermatological disorders, myasthenia gravis, glaucoma, multiple sclerosis (MS), autoimmune encephalomyelitis, and anticholinergic poisoning (e.g., overdose of anticholinergic drugs, such as certain antipsychotics, tricyclic antidepressants, and antihistamines). In addition, the use of reversible or pseudo-reversible AChE inhibitors has also been proposed in treating organophosphorous (OP) and nerve agent poisoning.

Accordingly, in some embodiments, the presently disclosed subject matter provides a method of treating or preventing a disease, disorder, or condition treatable or preventable via AChE inhibition in a subject in need thereof and/or of extending the lifespan of a subject. In some embodiments, the disease, disorder or condition treatable or preventable by AChE inhibition is selected from the group comprising a neurological disorder, a cognitive disorder, a dermatological disorder, myasthenia gravis, glaucoma, multiple sclerosis, autoimmune encephalomyelitis, OP poisoning, nerve agent poisoning, and anticholinergic poisoning. In some embodiments, the disease, disorder or condition treatable or preventable by inhibition of AChE is selected from the group comprising a dermatological disorder, myasthenia gravis, glaucoma, multiple sclerosis, autoimmune encephalomyelitis, OP poisoning, nerve agent poisoning, and anticholinergic poisoning.

In some embodiments, the method comprises administering to the subject an effective amount of at least one compound selected from the group comprising tilorone, a tilorone analog, cetylpyridinium, dequalinium chloride, bezedoxifene acetate, rifaximin, agelasine, and pharmaceutically acceptable salts thereof. In some embodiments, the at least one compound is tilorone or a tilorone analog and/or pharmaceutically acceptable salt thereof. Thus, in some embodiments, the method comprises administering to the subject an effective amount of tilorone or an analog and/or pharmaceutically acceptable salt thereof. In some embodiments, the tilorone analog or pharmaceutically acceptable salt is a selective inhibitor of AChE.

In some embodiments, the disease, disorder, or condition treatable or preventable by AChE inhibition is OP or nerve agent poisoning. Thus, in some embodiments, the subject is a subject in need of treatment for OP or nerve agent poisoning. In some embodiments, said subject is a subject suffering from OP or nerve agent poisoning (i.e., a subject known to have been exposed to an OP or nerve agent and, optionally, displaying one or more symptom of OP or nerve agent poisoning (e.g., headache, excessive sweating, muscle twitching, muscle weakness, convulsions, etc)); a subject suspected of having been exposed to OP or nerve agent poisoning and, optionally displaying one or more symptom of OP or nerve agent poisoning; or a subject at risk of OP or nerve agent poisoning (e.g., a subject living in or traveling to an area where pesticides are in use, such as on a farm; a subject living in an area at risk for a terrorist attack or in a war zone; or a soldier deploying to a war zone).

In some embodiments, the subject is a su bject at risk for OP or nerve agent poisoning and the subject is administered the at least one compound prior a potential exposure to an OP or a nerve agent, e.g., as a pre-treatment. In some embodiments, said pre-treatment is administered within about 3 days of the potential exposure (e.g., within about 2, 4, 6, 8, 10, 20 12, 18, 24, 30, 36, 42, 48 or 72 hours of the potential exposure).

In some embodiments, the subject is a subject suffering from OP or nerve agent poisoning or suspected of having OP or nerve agent poisoning and the at least one compound is administered after the subject's exposure or suspected exposure to the OP or nerve agent. In some embodiments, the at least one compound is administered within about 1 hour, within about 2 hours, within about 3 hours, within about 4 hours, within about 6 hours, within about 8 hours, within about 10 hours, within about 12 hours, within about 18 hours, within about 24 hours, or within about 48 hours of the subject's exposure or suspected exposure to the OP or nerve agent. In some embodiments, the at least one compound is administered within about 10 minutes to about 6 hours (e.g., within about 10, 15, 20, 30, 45, 60, 90, 120, 150, 180, 240, 300, or about 360 minutes) of the subject's exposure or suspected exposure to the OP or nerve agent.

In some embodiments, the method further comprises administering to the subject one or more additional treatment agents for OP poisoning or nerve agent poisoning. The one or more additional treatment agents for OP or nerve agent poisoning can be administered at about the same time as the at least one compound (e.g., the tilorone, tilorone analog or pharmaceutically acceptable salt thereof), prior to the at least one compound (e.g., one or more hours or days prior to the at least one compound), or after the at least one compound (e.g., one or more hours or days after the at least one compound). In some embodiments, the one or more additional treatment agents are selected from the group comprising atropine, pralidoxime or another oxime, diazepam or another benzodiazepine, and physostigmine salicylate. In some embodiments, the at least one compound is administered to the subject as a pre-treatment for possible exposure to an OP or nerve agent and the one or more additional treatment agents are administered after said exposure.

Accordingly, in some embodiments, the presently disclosed subject matter provides a method of treating or preventing OP or nerve agent poisoning wherein the method comprises administering a newly identified AChE inhibitor as disclosed herein or an analog or pharmaceutically acceptable salt thereof. In some embodiments, the newly identified AChE inhibitor is tilorone. In some embodiments, the method comprises administering a tilorone analog (e.g., another compound of Formula (I) or (II)) or a pharmaceutically acceptable salt thereof.

In some embodiments, the disease or condition treatable or preventable by inhibition of AChE is a dermatological disorder. In some embodiments, the dermatological disorder is selected from the group comprising a condition associated with Domodex brevis and/or Demodex folliculorum mites, a bacterial infection, acne, seborrheic dermatitis, perioral 20 dermatitis, acneform rash, transient acantholytic dermatosis, acne necrotica milliaris, steroid induced dermatitis, primary irritation dermatitis, and rosacea. In some embodiments, the dermatological disorder is other than a dermatological disorder caused by an autoimmune condition.

In some embodiments, the at least one compound has a 50% inhibitory concentration (IC₅₀) for AChE (e.g., human and/or eel AChE) of about nM or less (e.g., about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 15 nM or less, or about 10 nM or less). In some embodiments, the at least one compound has an IC₅₀ for AChE (e.g., human and/or eel AChE) between about 1 nM and about 100 nM (e.g., about 1, 5, 10, 15, 20, 25, 30, 35, 40, 45, 50, 55, 60, 65, 70, 75, 80, 85, 90, 95, or about 100 nM). In some embodiments, the at least one compound has an IC50 for human AChE of about 75 nM or less. In some embodiments, the at least one compound has an IC50 for human AChE of about 50 nM to about 75 nM. Alternatively or additionally, in some embodiments, the at least one compound has an IC₅₀ for eel AChE of about 15 nM or less (e.g., between about 1 nM and about 15 nM).

In some embodiments, the at least one compound (e.g., the tilorone, tilorone analog and/or pharmaceutically acceptable salt thereof) selectively inhibits AChE (e.g., human and/or eel AChE) compared to other cholinesterases. In some embodiments, the at least one compound selectively inhibits AChE (e.g., human or eel AChE) compared to butyrylcholinesterase (BuChE) (e.g., human and/or eel BuChE). In some embodiments, the at least one compound has an IC₅₀ for AChE (e.g., human or eel AChE) that is at least about 100 times lower than that compound's IC₅₀ for BuChE (e.g., human or eel BuChE). In some embodiments, the at least one compound's IC₅₀ for AChE (e.g., human or eel AChE) is at least about 200, 300, 400, 500, 600, 700, 800, or 900 times lower than its IC₅₀ for BuChE (e.g., human or eel BuChE). In some embodiments, the at least one compound has an IC₅₀ for AChE (e.g., human or eel AChE) that is at least about 1000 times lower than its IC₅₀ for BuChE (e.g., human or eel BuChE). In some embodiments, the at least one compound has an IC₅₀ for AChE (e.g., human or eel AChE) that is about 1000, about 1100, about 1200, or about 1300 times lower than its IC₅₀ for BuChE (e.g., human or eel BuChE).

In some embodiments, the at least one compound (e.g., the tilorone or tilorone analog and/or pharmaceutically acceptable salt thereof) binds (e.g., reversibly binds) in the active site gorge of the AChE. In some embodiments, the at least one compound (e.g., the tilorone or tilorone analog and/or pharmaceutically acceptable salt thereof) exhibits pi-pi interactions with W286 of the peripheral anionic site (PAS) of human AChE (hAChE). In some embodiments, the at least one compound (e.g., the tilorone or tilorone analog and/or pharmaceutically acceptable salt thereof) exhibits pi-pi interactions with Y341 of hAChE. In some embodiments, the at least one compound is also an agonist of the alpha7 nicotinic acetylcholine receptor (α7 nAChR).

In some embodiments, the subject is a warm-blooded animal, e.g. a mammal or bird. In some embodiments, the subject is a mammal. In some embodiments, the subject is a human.

In some embodiments, the administering is performed via oral administration, intravenous (IV) administration, intraperitoneal (IP) administration, topical administration, intracerebroventricular (ICV) administration, or intrathecal (IT) administration.

In some embodiments, the at least one compound is tilorone or a tilorone analog and/or pharmaceutically acceptable salt thereof and the tilorone or tilorone analog and/or pharmaceutically acceptable salt thereof has a structure of one of Formula (I) and Formula (II):

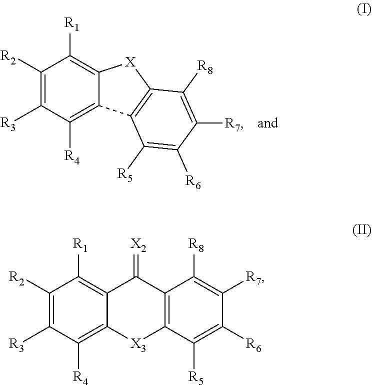

wherein [symbol] is absent or a single bond; X is selected from the group comprising —C(═Z)—, —S(═O)₂—, —CH₂—, —O—, —S—, and —NH—; Z is selected from O, S, and CH₂; X₂ is selected from O, S, and CH₂; X₃ is selected from —C(═Z)—, —CH₂—, —O—, —S—, and —NH—; each of R₁, R₂, R₃, R₄, R₅, R₆, R₇, and R₈ is independently selected from the group comprising H, alkyl, amino, hydroxy, alkoxy, and —X₄-L-N(R₉)₂; X₄ is selected from —O—, —S—, —NH—C(═O)—, —O—C(═O)—, —C(═O)—, —C(OH)—and —C(═O)—O—; L is a bivalent linker moiety (e.g., an alkylene, arylene, or aralkylene group), optionally a C₁-C₆ alkylene group; and each R₉ is alkyl, optionally C₁-C₈ or C₁-C₆ alkyl; aralkyl, or aryl, or wherein two R₉ together form a cyclic bivalent group (e.g., so that the two R₉ groups together with the nitrogen atom to which they are attached from a ring, such as a pyrrolidine, pyrrole, pyridine, piperidine, morpholine, or diazinane group); or a pharmaceutically acceptable salt thereof.

In some embodiments, at least one of R₁-R₈ comprises a tertiary amino group, e.g., is a group of the formula —X₄-L-N(R₉)₂. In some embodiments, at least one of R₂, R₃, R₆, and R₇ comprises a tertiary amino group, e.g., is a group of the formula —X₄-L-N(R₉)₂. In some embodiments, one of R₁-R₄ comprises a tertiary amino group (e.g., is a group of the formula —X₄-L-N(R₉)₂) and one of R₅-R₈ comprises a tertiary amino group (e.g., is a group of the formula X₄-L-N(R₉)₂). In some embodiments, one of R₂ and R₃ comprises a tertiary amino group (e.g., is a group of the formula —X₄-L-N(R₉)₂) and one of R₆ and R₇ comprises a tertiary amino group (e.g., is a group of the formula X₄-L-N(R₉)₂). In some embodiments, X₄ is selected from —O— and —C(═O)—. In some embodiments, L is a C₁-C₆ alkylene (e.g., methylene, ethylene, propylene, butylene, pentylene, hexylene). In some embodiments, each R₉ is alkyl. In some embodiments, each R₉ is methyl or ethyl.

In some embodiments, one or two of R₁-R₈ is hydroxy. For example, in some embodiments, the tilorone analog is 2,7-dihydroxyfluoren-9-one.

In some embodiments, the tilorone analog is a compound of Formula (II). In some embodiments, X₃ is —CH₂— or —C(═O)—. In some embodiments, X₂ is 0.

In some embodiments, the tilorone analog is a compound of Formula (I). In some embodiments, [symbol] is absent and X is —C(═Z)— (e.g., —C(═O)—). In some embodiments, [symbol] is present (e.g., is a single bond) and X is selected from —C(═Z)—, —S(═O)₂—, —CH₂—, —O—, —S—, and —NH—. In some embodiments, X is selected from —C(═O)—, —C(═CH₂)—, —CH₂—, and —O—.

In some embodiments, the method further comprises administering to the subject one or more additional therapeutic agents, e.g., an agent known in the art for treating or preventing the neurological disorder, cognitive disorder, dermatological disorder, myasthenia gravis, glaucoma, MS, autoimmune encephalomyelitis, OP poisoning, nerve agent poisoning, or anticholinergic poisoning, or a symptom thereof.

In some embodiments, the presently disclosed subject matter provides a pharmaceutical composition for use in treating or preventing a disease, disorder, or condition treatable or preventable via AChE inhibition and/or for use in extending the lifespan of a subject. In some embodiments, the pharmaceutical composition comprises at least one compound selected from the group comprising tilorone, a tilorone analog, cetylpyridinium, dequalinium chloride, bezedoxifene acetate, rifaximin, agelasine, and pharmaceutically acceptable salts thereof. In some embodiments, the pharmaceutical composition is for use in treating or preventing a disease, disorder or condition treatable or preventable via AChE inhibition. In some embodiments, said disease, disorder or condition is a neurological or cognitive disorder. In some embodiments, said disease, disorder, or condition is a dermatological disorder, myasthenia gravis, glaucoma, multiple sclerosis, autoimmune encephalomyelitis, OP poisoning, nerve agent poisoning, or anticholinergic poisoning. In some embodiments, the disease, disorder, or condition is a dermatological disorder. In some embodiments, the disease, disorder or condition is OP, nerve agent, or anticholinergic poisoning.

### V. Salts and Pharmaceutical Compositions

As noted above, in some embodiments, the active compounds of the presently disclosed subject matter (i.e., the AChE inhibitors, such as tilorone or an analog thereof) can be provided as pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′- dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

The active compounds can be used on a sample either in vitro (for example, on isolated cells or tissues) or in vivo in a subject (i.e. living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans and non-human primates, as well as other mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

In some embodiments, the active compounds can include more than one of the active compounds described herein or can include one or more of the active compounds and one or more additional therapeutic agents. Thus, in some embodiments, the active compound or compounds can be administered along with one or more additional therapeutic agents known in the art for treating a disease, disorder or condition treatable by AChE inhibition. The active compounds and the one or more additional therapeutic agents can be provided in a single formulation or co-administered in separate formulations at about the same time or at different times (e.g., different times within the same day, week, or month).

In some embodiments, the active compound of the presently disclosed subject matter can be administered in a pharmaceutically acceptable composition where the compound can be admixed with one or more pharmaceutically acceptable carriers. The term “pharmaceutically acceptable carrier” means a non-toxic material that does not interfere with the effectiveness of the biological activity of the active ingredients. In some embodiments, the pharmaceutically acceptable composition can also contain salts, buffering agents, preservatives, compatible carriers, and optionally other therapeutic agents.

Suitable methods for administration of an active compound or pharmaceutically acceptable composition thereof to a subject include, but are not limited to intravenous injection, oral administration, buccal, topical, subcutaneous administration, intraperitoneal injection, pulmonary, intanasal, intracranial injection, and rectal administration. The particular mode of administering a composition matter depends on various factors, including the distribution and abundance of cells to be treated and mechanisms for metabolism or removal of the composition from its site of administration.

An effective dose of a composition of the presently disclosed subject matter is administered to a subject. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration. In some embodiments, the active compounds can be used in dosages from 0.001-1000 mg/kg body weight.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient;

and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder. The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the active compound or compounds of the presently disclosed subject matter can include an agent which controls release of the compound, thereby providing a timed or sustained release compound.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

Chemicals and Reagents

Tilorone dihydrochloride (Item No. 17868) was purchased from Cayman Chemical (Ann Arbor, Michigan, United States of America) The MicroSource Spectrum screening compound library (MicroSource Discovery Systems, Inc., Gaylordsville, Connecticut, United States of America) was a generous gift from Dr. Ethan Perlstein.

### Assay Central®

Software available under the registered trademark ASSAY CENTRAL® (Collaborations Pharmaceuticals Inc., Raleigh, North Carolina, United States of America) was developed from open source descriptors and algorithms that have been previously describee⁴⁰⁻⁴⁹ and combined with additional proprietary scripts. Structure-activity datasets were collated in Molecular Notebook (Molecular Materials Informatics, Inc.; Montreal, Canada) and thoroughly curated to generate a Bayesian machine learning model with multiple scripts. A series of rules was employed to detect problematic data, corrections were implemented by a combination of automated and human re-curation for structure standardization. This approach produces a high-quality dataset and a Bayesian model to predict activities for proposed compounds. These Bayesian models utilize extended-connectivity fingerprints of maximum diameter 6 (ECFP6) descriptors generated from the Chemistry Development Kit library⁵⁰. These descriptors have widely been noted for their ability to map structure-activity relationships⁵¹. From all training set molecules, the ASSAY CENTRAL® software enumerates all fingerprints from the training set and determines a given fingerprint's contribution to a binary activity classification from the ratio of its presence in active and inactive molecules. ASSAY CENTRAL® software uses the summation of these contributions for a given molecule to produce a probability-like score. Metrics such as Receiver Operator Characteristic (ROC), Recall, Precision, F1 Score, Cohen's Kappa and Matthew's Correlation Coefficient are generated from internal five-fold cross-validation of the model. To maximize these internal performance statistics, the software can select a reasonable activity threshold, and generate predictions as well as applicability scores for any desired compound. Higher prediction scores are desirable as scores higher than 0.5 are assigned to active compounds (inhibitors). Higher applicability scores are also desirable as it ensures the representation of the drug in the training set⁵¹. ASSAY CENTRAL® software has been used in various drug discovery projects and the applicability of the model statistics have also been previously described^{40-48, 52}.

Curation of Training Datasets

All three Bayesian models built in this study were based on ChEMBL datasets using all IC₅₀ data for ChEMBL 220 (human AChE), ChEMBL 4078 (Electrophorus electricus (eel) AChE), and ChEMBL 1914 (human butyrylcholinesterase (BuChE)). Any molecules that did not contain IC₅₀ data were removed from the training set. Any salts or minor components were removed through a feature of ASSAY CENTRAL® software that allows for the retention of major components only. ASSAY CENTRAL® software was also used to calculate the threshold of the human BuChE and AChE models while a threshold of 100 nM was manually set for the eel AChE model.

Subvalidations

The performance of the human and eel AChE models in predicting the AChE experimental data (see below) was assessed through external cross-validation metrics termed “Subvalidations.” Any compounds that overlapped between the training and testing sets were removed by the testing set resulting in a 4545-compound test set for AChE eel and a 4601-compound test set for human AChE. The threshold of activity for this test sets were set at 50% inhibition. ROC, precision, recall and specificity metrics were used to compare the results of these subvalidations.

Comparison Between Additional Machine Learning Algorithms

The extended-connectivity fingerprints (ECPF6) molecular descriptor that ASSAY CENTRAL® software utilizes was also exploited by multiple machine learning algorithms to build models for the eel and human AChE and human BuChE. The algorithms included random forest, k-Nearest Neighbors, support vector classification, naive Bayesian, AdaBoosted decision trees, and deep learning. Further details of these machine learning methods have been reported previously by us and others^{45, 46, 49}.

Activity Assays

Eel AChE Assays

A total of 2558 compounds from The MicroSource Spectrum Collection (MicroSource Discovery Systems, Inc., Gaylordsville, Connecticut, United States of America) were tested in a single-point format at an initial concentration of 20 μM using the fluorescence assay kit sold under the tradename AMPLEX™ Red Acetylcholine/Acetylcholinesterase Assay Kit (Product No. A12217; ThermoFisher Scientific, Waltham, Massachusetts, United States of America). 127 molecules were excluded from the final analyses due to the appearance of poor solubility (precipitation) in the library, leading to unknown concentrations. Enzymatic reactions consisted of 50 μM acetylcholine, 200 μM AIVIPLEX™ Red reagent, 1 U/mL horseradish peroxidase, 0.1 U/mL choline oxidase and 0.096 U/mL acetylcholinesterase, with 30 1% DMSO. Reactions were performed in black 384-well plates (product number 3820; Corning Inc., Corning, New York, United States of America), incubated at room temperature for 45 minutes, and read on a microplate reader sold under the tradename SPECTRAMAX™ iD5 (Molecular Devices, San Jose, California, United States of America) with an excitation/emission pair of 560/590 nm. Control reactions lacking acetylcholinesterase but containing 5 μM H₂O₂ in presence of compounds were analyzed at the same wavelength to exclude compounds that display assay interference. Dose-response curves were performed in duplicate.

Human AChE Assays

Tilorone (hydrochloride) was tested for human AChE activity using the Acetylcholinesterase Inhibitor Screening Kit (Colorimetric) (product number K197-100; BioVision, Inc., Milpitas, California, United States of America). Reactions were performed in a total volume of 100 μL. Briefly, 2.5 μL of 1:50 diluted enzyme and 2.5 μL 1:12 diluted substrate were used per reaction with 1% DMSO in 384-well clear plates (product number 781162; Greiner BioOne, GmbH, Kremsmunster, Austria). Kinetic reactions were recorded for 40 minutes at room temp using a microplate reader sold under the tradename SPECTRAMAX™ iD5 (Molecular Devices, San Jose, California, United States of America) and an absorbance of 412 nm. Percent inhibition was calculated by choosing two time points in the linear range of the reaction for the positive control (PC), and comparing the slope of the reaction (slope=(OD₂-OD₁)/(t₂-t₁)) to the slope of a reaction containing sample compound (5): % Relative inhibition=(Slope_{PC}−Slope_{S})/Slope_{PC}x100.

Secondary testing of tilorone against human AChE was performed by Eurofins Discovery (Eurofins Cerep SA, Le Bois L′évêque, France), using a photometric assay employing acetylthiocholine as a substrate for AChE to produce thiocholine, which reacts with 5:5-dithiobis-2-nitrobenzoate to produce the yellow product 5-thio-2-nitrobenzoic acid⁵³.

Human BuChE Assay

Tilorone was tested for butyrylcholinesterase activity using the Butyrylcholinesterase Activity Kit (Colorimetric) (product number K516; BioVision, Inc., Milpitas, California, United States of America). Reactions were performed in 384-well plates (781162, Greiner BioOne, GmbH, Kremsmunster, Austria) with 1% DMSO, according to the manufacturer's instructions with a few modifications. Compounds were incubated with enzyme for 30 minutes at room temperature before addition of substrate. Total volume of the reaction was 100 μL, we use 5 μL enzyme and kinetic reactions were performed at 30° C. for 60 minutes using a microplate reader sold under the tradename SPECTRAMAX™ iD5 (Molecular Devices, San Jose, California, United States of America) and an absorbance of 412 nm. Percent activity was calculated by calculating the slope of each reaction as stated above and comparing the positive control (PC) to sample (S): % Relative activity=Slope_{S}/Slope_{PC}x100. We evaluated if assay reagents could interfere with the absorbance by adding compound or DMSO to a 150 μM solution of 5-thio-2-nitrobenzoic acid and measuring at 412 nM.

Docking

Tilorone was docked in the human AChE crystal structure (PDB code 4EY7). The docking sphere was centralized from the position of the crystallized ligand donepezil with a diameter of 9.85 Å. CHARMm based docking (CDOCKER)⁵⁴ was used to generate a maximum of 10 poses of tilorone within this sphere using ridged docking. CDOCKER, a simulated annealing-based docking algorithm, was run within software available under the tradename DISCOVERY STUDIO® (Biovia, San Diego, California, United States of America) using the following default parameters: 1000 dynamic steps, which included electrostatic interactions, 2000 heating steps, 5000 cooling steps and a final cooling target temperature of 300K. The energy of the top scoring CDOCKER pose (CDOCKER interaction energy score=62.99) was calculated following an in-situ ligand minimization step to be −290.50 kcal/mol and including ligand entropic energy using the default setting in the DISCOVERY STUDIO® software. As a reference, the binding energy for donepezil was calculated to be −125.189 kcal/mol. In order to generate a CDOCKER score for the control, donepezil was docked in the same sphere and the score with the lowest RMSD (0.426 Å) from the crystalized ligand had a CDOCKER interaction energy score of 54.96.

Safety Profiling

Safety profiling of tilorone was performed by Eurofins Discovery (Eurofins Cerep SA, Le Bois L′évêque, France) using the SafetyScreen44 panel of in vitro pharmacology assays to assess binding or uptake of tilorone by 44 different targets of interest, including kinases, GPCRs, transporters, ion channels, nuclear receptors and other non-kinase enzymes.

Statistics

Statistical analysis for dose-response curves was performed using software available under the tradename GRAPHPAD™ Prism 8 (GraphPad Software, San Diego, California, United States of America). A non-linear regression log(inhibitor) vs. response equation with three or four parameters were used (indicated in legend), and the Hill slopes for each of the graphs are reported.

### Example 1

### Machine Learning Models

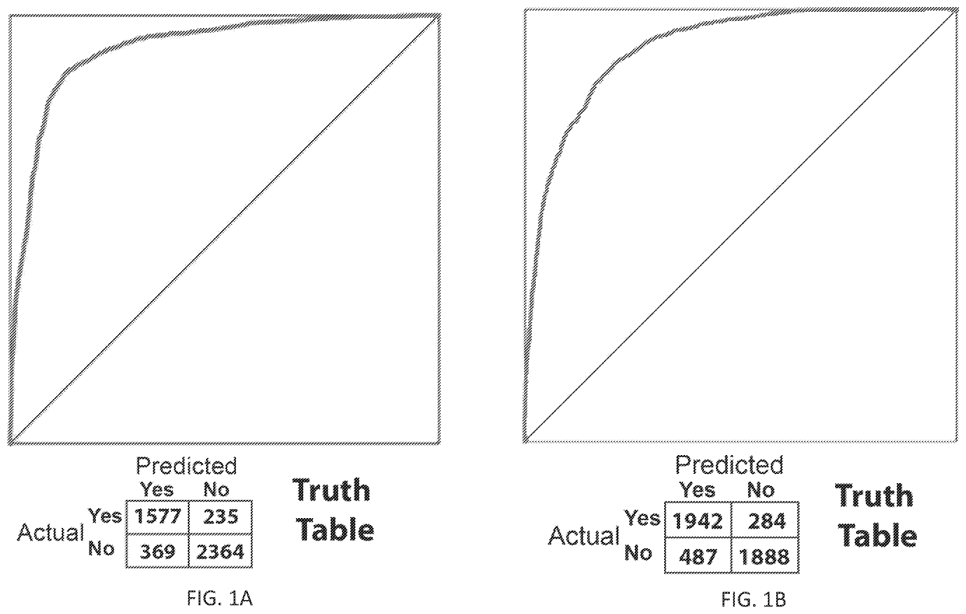

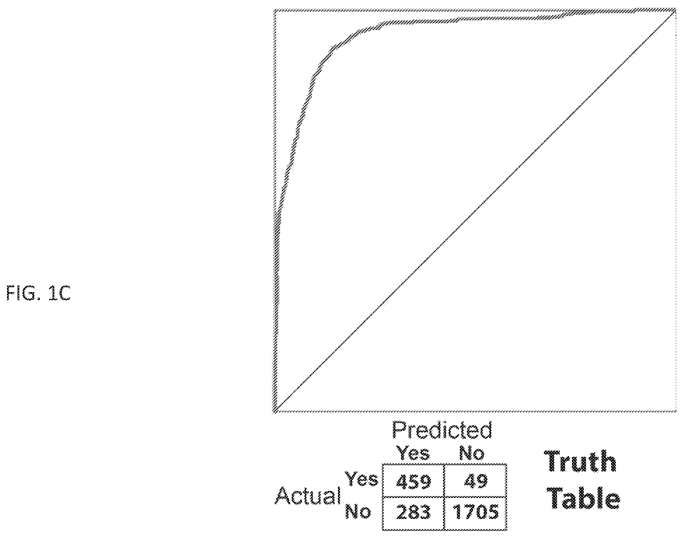

The threshold of activity for the eel AChE model was manually set to be 100 nM resulting in model with 4545 compounds and 1812 active molecules. ROC, precision, recall, and specificity were 0.923, 0.810, 0.870, and 0.865 respectively. See FIG. 1A. The human AChE model was built with 4601 compounds, 2226 of which were active and had a calculated threshold of 1.98 μM. Internal five-fold cross-validation statistics were excellent and resulted in values of 0.911, 0.800, 0.872, 0.795 for ROC, precision, recall and specificity respectively.

See FIG. 1B. The human BuChE model was smaller based on IC₅₀ data from 2496 compounds with only 508 active compounds and a calculated threshold of 112 nM, however, the internal validation metrics were also excellent with ROC of 0.938, precision of 0.619, recall of 0.904, and specificity of 0.858. See FIG. 1C.

Comparison Between Additional Machine Learning Algorithms

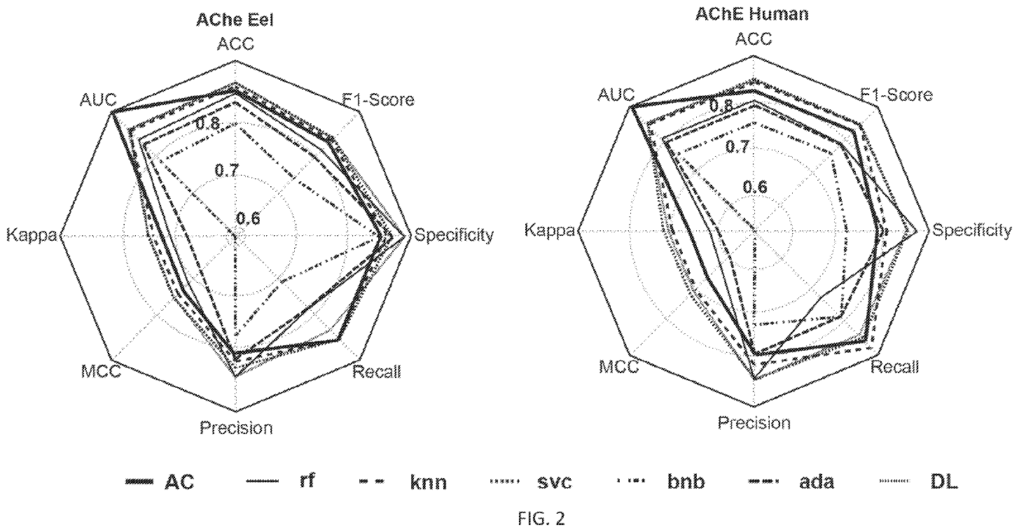

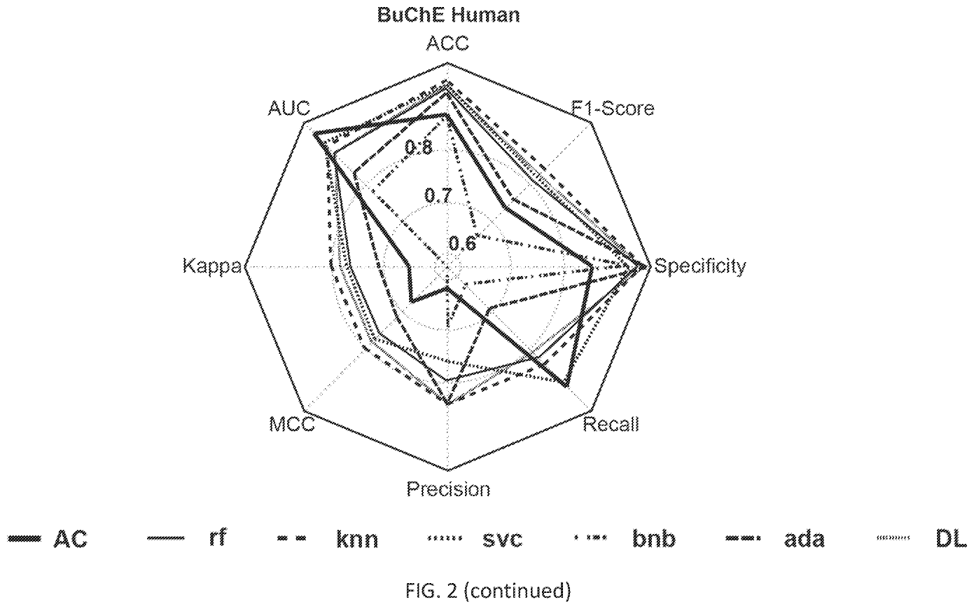

Very similar model statistics were observed across machine learning models for the three datasets when using either ASSAY CENTRAL®, random forest, k-Nearest Neighbors, support vector classification, naïve Bayesian, AdaBoosted decision trees, and deep learning. See FIG. 2.

A machine learning model was used to predict potential AChE inhibitors from the MicroSource Spectrum collection; a library composed of 8 plates containing 320 compounds each. Initially, an activity screen was performed on the plate with the largest number of compounds with predicted AChE activity, however none of the compounds reached the 50% inhibition threshold set for activity. The remaining compounds in the library were then screened, discarding those that displayed solubility issues.

### Example 2

### Eel Screen

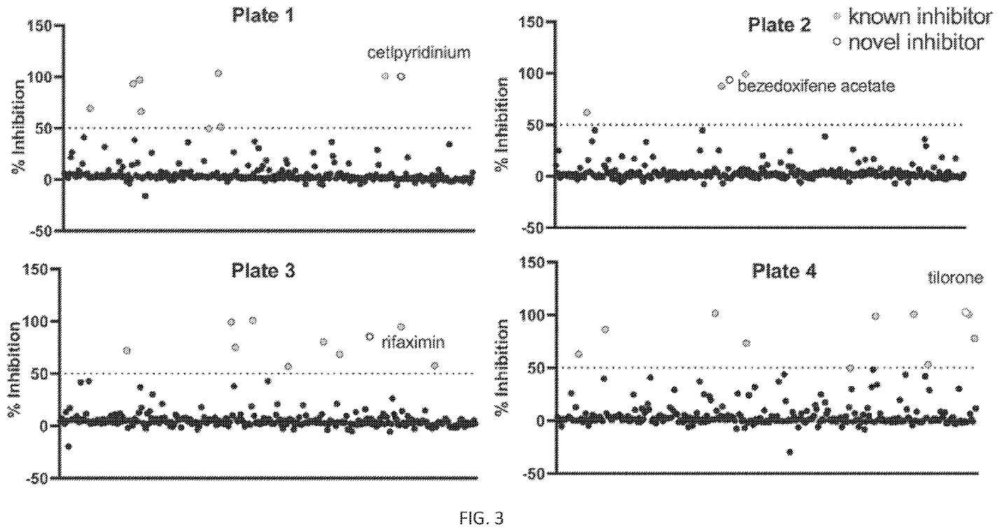

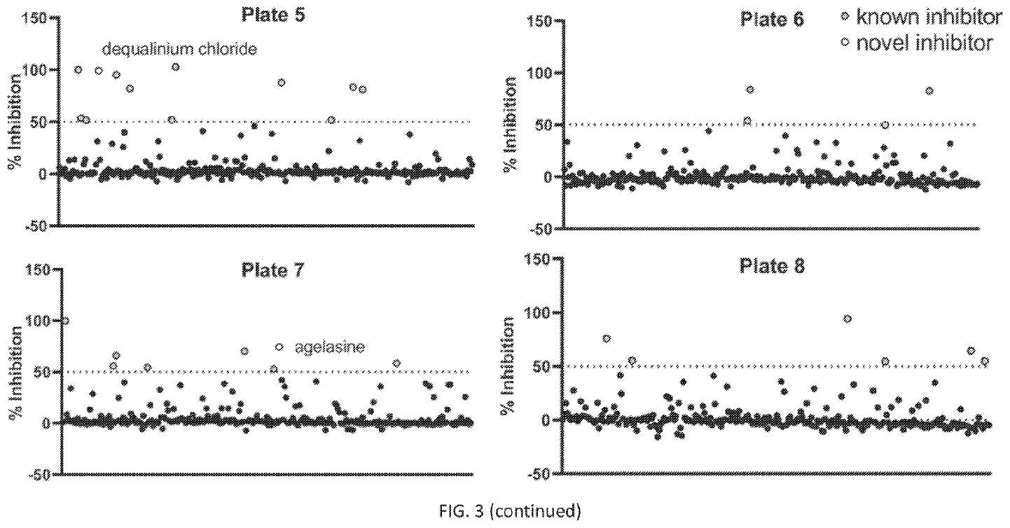

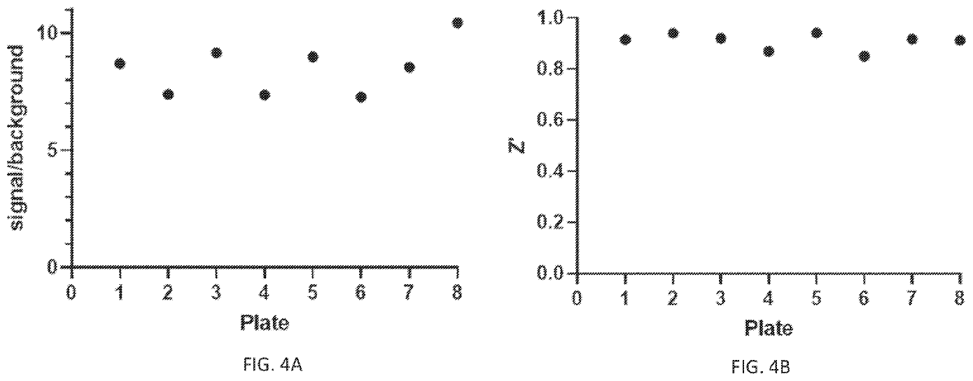

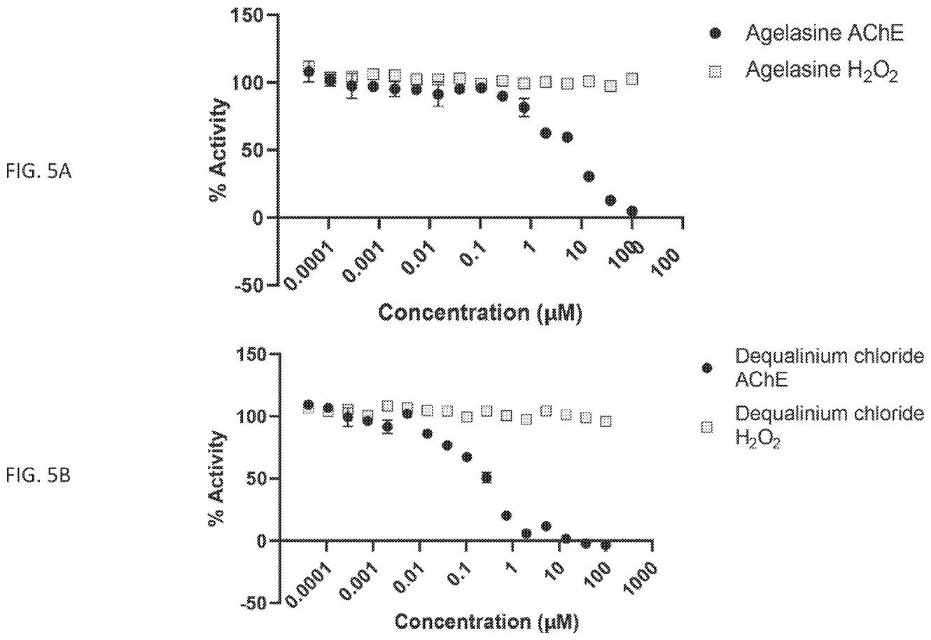

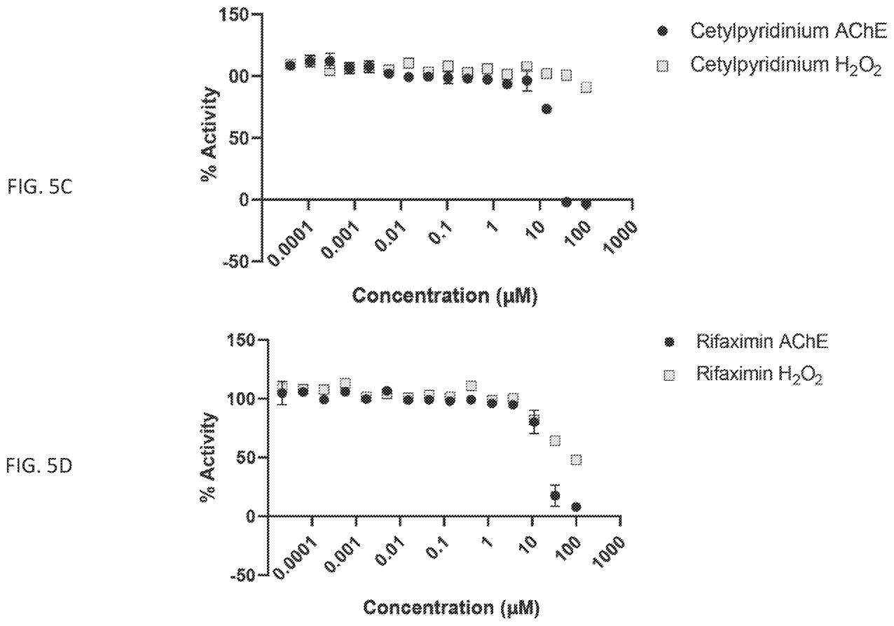

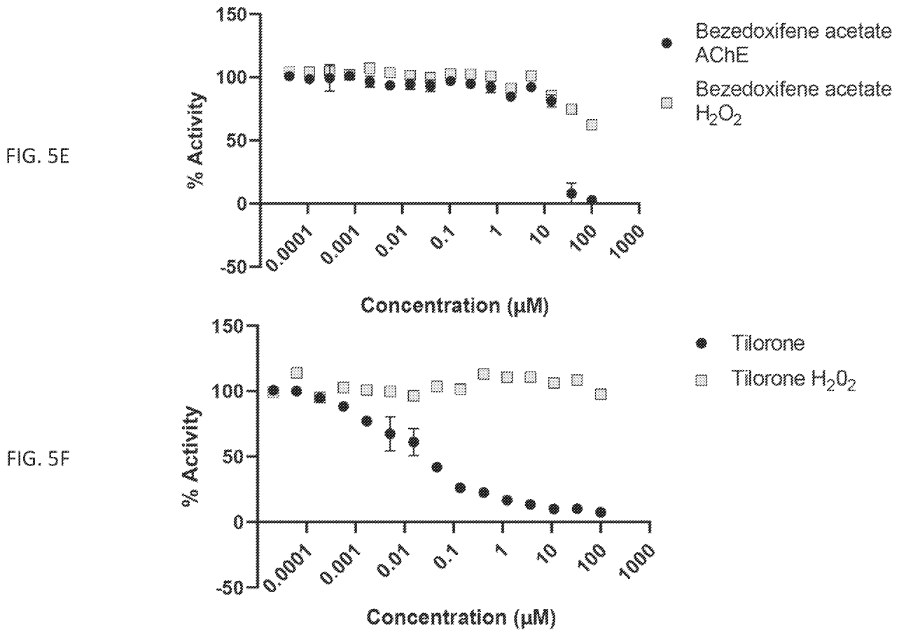

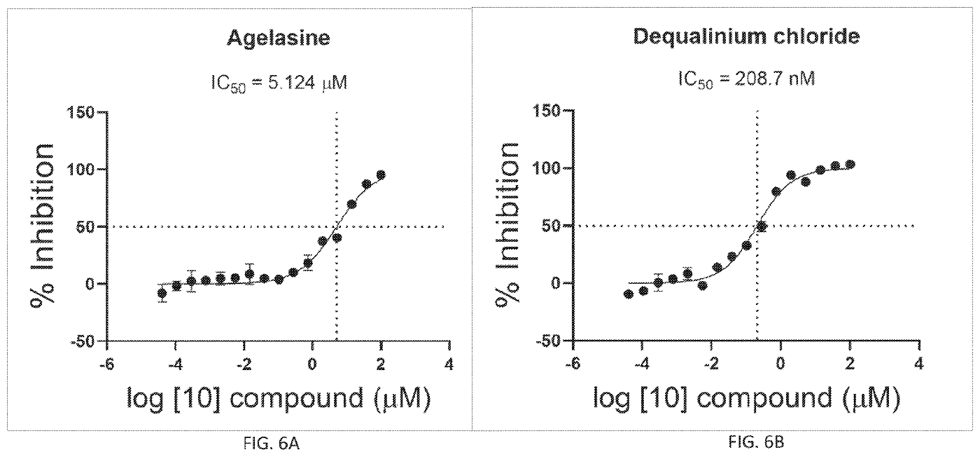

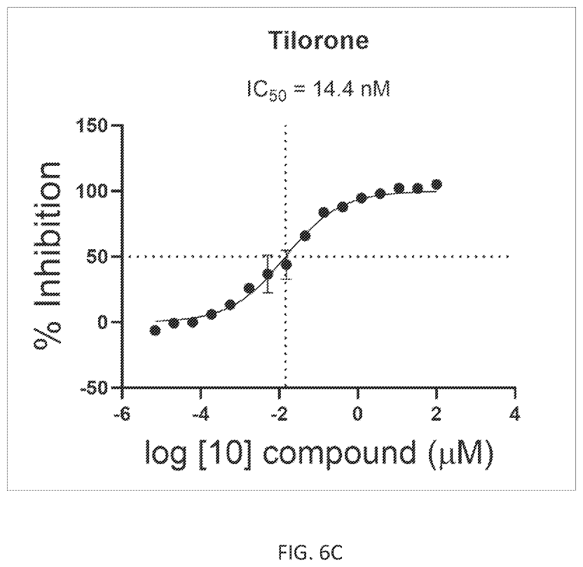

Through the high throughput screen of 2431 compounds, 66 compounds were identified as “active” against eel acetylcholinesterase, as defined by a predetermined cutoff of inhibiting enzyme activity ≥50%. See FIG. 3. The average signal to background ratio of 8.49±1.10 and an average Z′ of 0.91±0.03. See FIGS. 4A and 4B. Some of these compounds are known inhibitors of acetylcholinesterase. However, some of the compounds, including agelasine, dequalinium chloride, cetylpyridinium, rifaximin, bezedoxifene acetate, and tilorone, are believed to be identified here as AChE inhibitors for the first time. The newly identified inhibitors were tested for assay interference, by performing dose-response curves with compounds in the presence of the reaction product, H₂O₂. Agelasine, dequalinium chloride and tilorone appeared to show a dose-response reaction to the enzyme without a corresponding dip in an enzyme-free H₂O₂ reaction; therefore, these compounds likely represent true inhibition of the enzyme. See FIGS. 5A-5F. Analysis of these data revealed three compounds with IC₅₀ values in sub-micromolar to low micromolar range. See FIGS. 6A-6C. Tilorone was the most potent of these, with an IC₅₀ of 14.4 nM for the eel acetylcholinesterase.

### Example 3

### Human In Vitro Assays

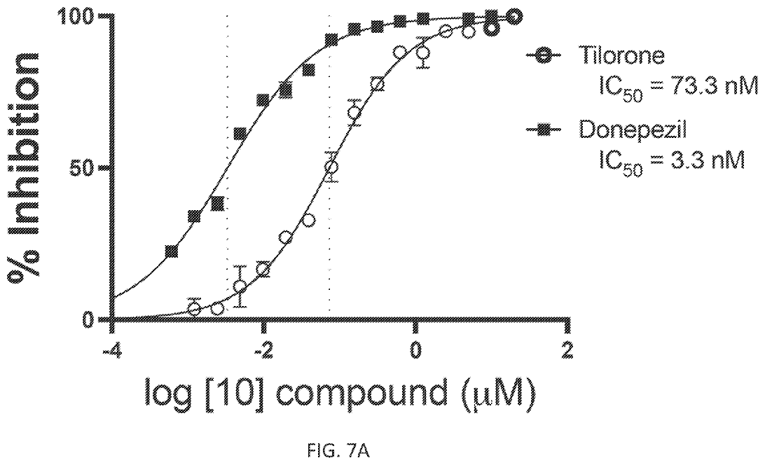

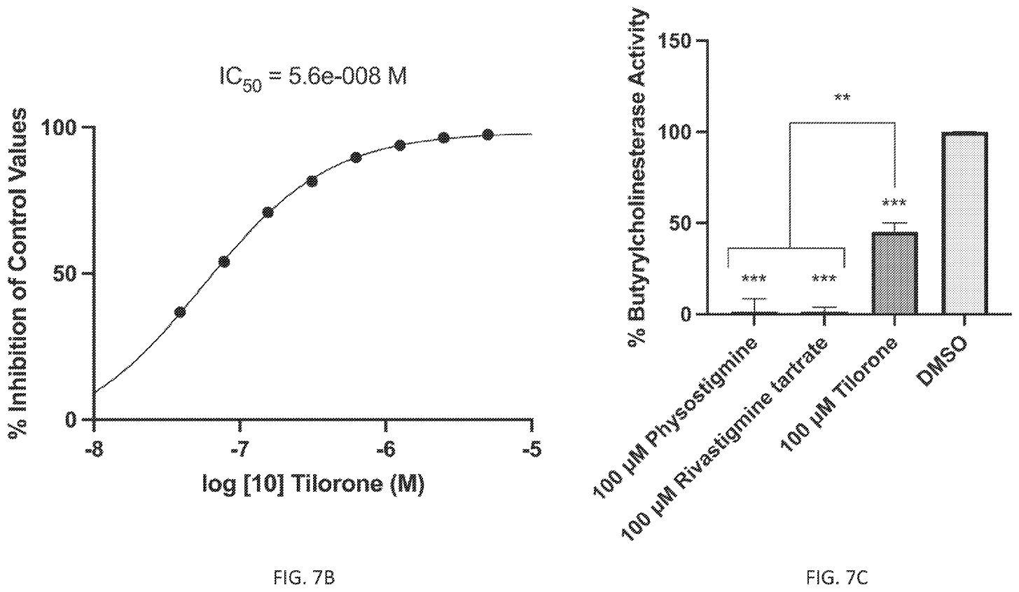

The ability of tilorone to inhibit human AChE was investigated initially using the Colorimetric Acetylcholinesterase inhibition assay. Tilorone displayed an IC₅₀ of 73.3 nM against the human AChE, which was less potent compared to the potent AChE inhibitor donepezil. See FIG. 7A. This activity was also verified through testing by a third-party (Eurofins Discovery), which reported an IC₅₀ of 56 nM for tilorone against human AChE (see FIG. 7B), which was ten times more potent than the 580 nM reported for the approved drug galantamine used as a positive control for this assay. A new stock of tilorone was purchased for these and all subsequent assays compared with that used in the initial eel AChE screen.

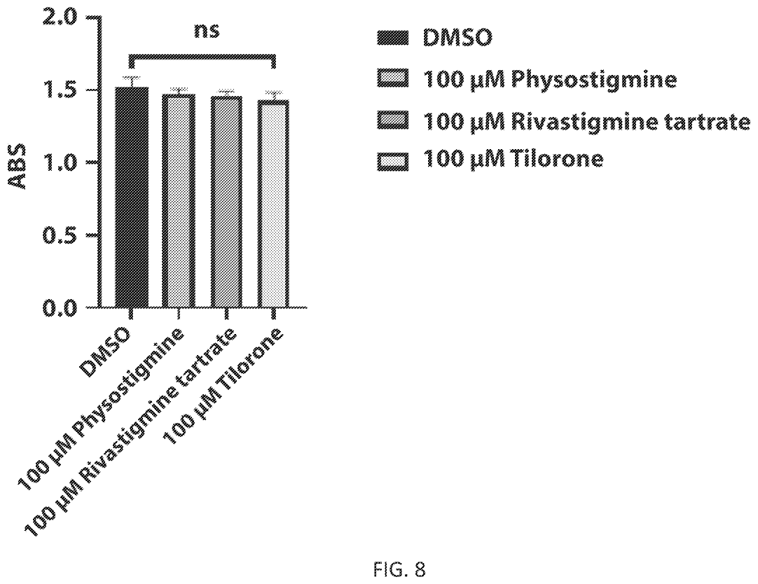

AChE has a “sister” cholinesterase, butyrylcholinesterase (BChE), that also hydrolyzes acetylcholine and shares 51.6% amino acid sequence identity with AChE 55. To investigate this specificity, tilorone was tested against human BChE. Compared to rivastigmine, tilorone was a very weak inhibitor of BChE, displaying only 50% inhibition at 100 μM. See FIG. 7C. Control with compound and buffer showed that this signal was not due to absorbance of tilorone at 412 nM. See FIG. 8.

### Example 4

### Validating ASSAY CENTRAL® Models

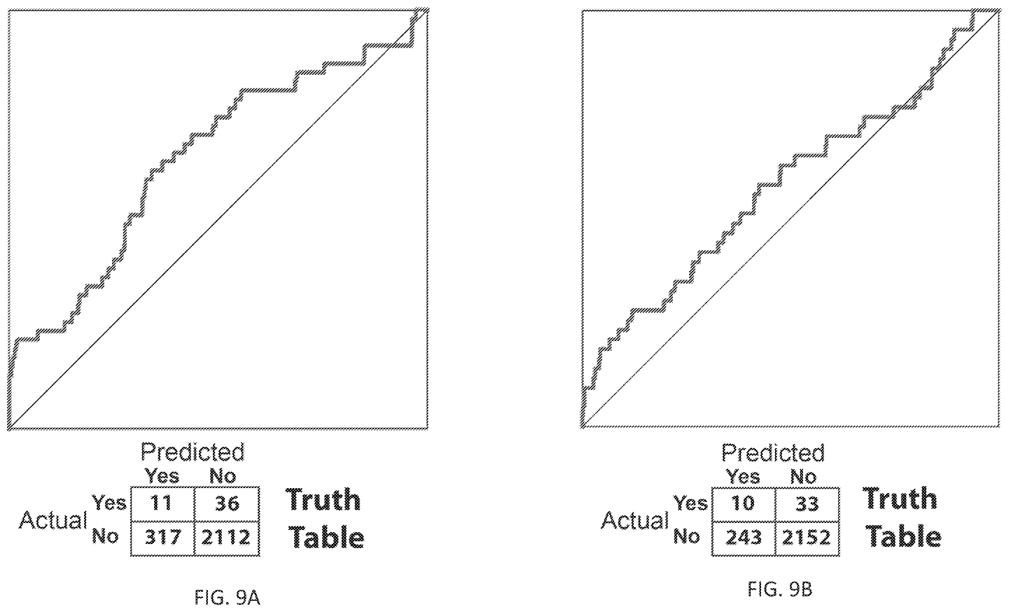

The screening data generated for the eel and human AChE was used as external test sets for the ASSAY CENTRAL® Machine learning models generated from published data. The ROC plots demonstrate the performance of the models in predicting the eel experimental data. The external five-fold cross-validation metrics are very similar with slightly better ROC for the eel AChE model (0.647 vs. 0.581). See FIGS. 9A and 9B.

### Example 5

### Docking Studies

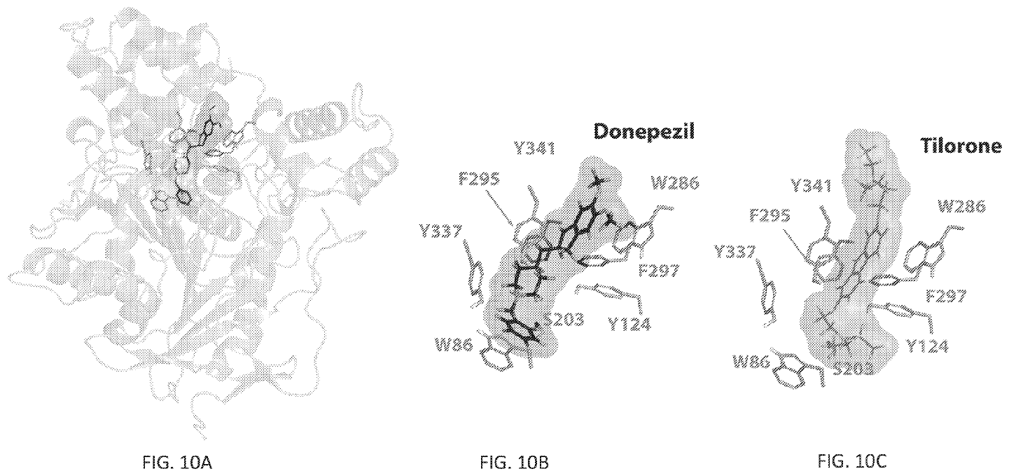

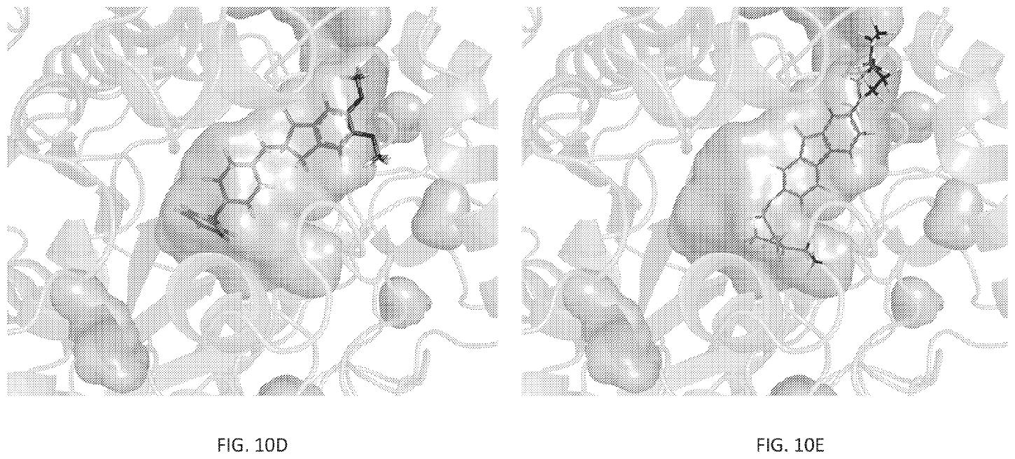

Tilorone was docked in silico into recombinant human acetylcholinesterase (rhAChE) using the scaffold of rhAChE crystallized with donepezil (PDB code 4EY7). See FIG. 10A. Like donepezil, tilorone appears to inhibit AChE activity through occupation of the majority of the active site gorge, and perhaps also through similar interaction W286 in the peripheral anionic site. See FIGS. 10B and 10C. Unlike donepezil, however, it appears that tilorone can display pi-interactions with Y341 through is core ring structure. It is important to note that since the docking studies were performed using a donepezil-bound crystal structure, the “swinging gate” of Y341 is in the open conformation for this simulation. Donepezil is 30 times more selective for AChE than BChE⁵⁶, and if tilorone is inhibiting AChE activity using a similar mechanism, it would follow that tilorone would not inhibit BChE. One of the crucial interactions for BChE inhibition is with W82 [31347933] (W86 human AChE numbering), an interaction that donepezil shows, but not tilorone. See FIGS. 10D and 10E.

### Example 6

### Safety Screening

In vitro safety pharmacology profiling was performed for tilorone (Eurofins Discovery) using the SafetyScreen44™ panel. This panel included 38 binding assays and six functional assays of well-established targets and pathways that lead to off-target adverse drug reactions. Tilorone was tested at 1 μM against these targets and it was found to only inhibit one target >50% activity, namely acetylcholinesterase. See Table 1, below.

TABLE 1
Safety Screen of Tilorone at 1 μM.

| | % Inhibition of Specific |
|---|---|
| Binding Assays | Control Binding |
| A2A (h) (agonist radioligand) | −4 |
| alpha 1A (h) (antagonist radioligand) | 26 |
| alpha 2A (h) (antagonist radioligand) | 0 |
| beta 1 (h) (agonist radioligand) | 5 |
| beta 2 (h) (antagonist radioligand) | −6 |
| BZD (central) (agonist radioligand) | −9 |
| CB1 (h) (agonist radioligand) | 0 |
| CB2 (h) (agonist radioligand) | −16 |
| CCK1 (CCKA) (h) (agonist | −5 |
| radioligand) | |
| D1 (h) (antagonist radioligand) | −10 |
| D2S (h) (agonist radioligand) | 1 |
| ETA (h) (agonist radioligand) | 5 |
| NMDA (antagonist radioligand) | −10 |
| H1 (h) (antagonist radioligand) | −6 |
| H2 (h) (antagonist radioligand) | −11 |
| MAO-A (antagonist radioligand) | 3 |
| M1 (h) (antagonist radioligand) | 28 |
| M2 (h) (antagonist radioligand) | 26 |
| M3 (h) (antagonist radioligand) | 17 |
| N neuronal alpha 4beta 2 (h) (agonist | 0 |
| radioligand) | |
| delta (DOP) (h) (agonist radioligand) | 0 |
| kappa (h) (KOP) (agonist radioligand) | 4 |
| mu (MOP) (h) (agonist radioligand) | −4 |
| 5-HT1A (h) (agonist radioligand) | 12 |
| 5-HT1B (h) (antagonist radioligand) | 21 |
| 5-HT2A (h) (agonist radioligand) | 25 |
| 5-HT2B (h) (agonist radioligand) | −9 |
| 5-HT3 (h) (antagonist radioligand) | 6 |
| GR (h) (agonist radioligand) | −5 |
| AR(h) (agonist radioligand) | −4 |
| V1a (h) (agonist radioligand) | −1 |
| Ca2+ channel (L, dihydropyridine site) | 4 |
| (antagonist radioligand) | |
| Potassium Channel hERG (human)- | −4 |
| [3H] Dofetilide | |
| KV channel (antagonist radioligand) | 0 |
| Na+ channel (site 2) (antagonist | 1 |
| radioligand) | |
| norepinephrine transporter (h) | 14 |
| (antagonist radioligand) | |
| dopamine transporter (h) (antagonist | −9 |
| radioligand) | |
| 5-HT transporter (h) (antagonist | 44 |
| radioligand) | |

| | % Inhibition of |
|---|---|
| Enzyme and cell-based assays | Control Values |
| COX1(h) | −17 |
| COX2(h) | −1 |
| PDE3A (h) | 2 |
| PDE4D2 (h) | −15 |
| Lck kinase (h) | 13 |
| acetylcholinesterase (h) | 92 |

### Discussion of Examples 1-6

Acetylcholine is a neurotransmitter at neuromuscular junctions and at synapses in the autonomic and central nervous systems however, it also functions as a signaling molecule in non-neuronal contexts related to cellular functions, such as proliferation and differentiation, as well as performing organ functions, like wound healing in skin or mucus production in lungs⁵⁷. The cholinergic hypothesis of AD argues that the cognitive decline associated with the disease is due to the gradual loss of these cholinergic neurons, and three of the five FDA-approved treatments for AD are cholinesterase inhibitors. There is also some evidence that AChE can help in aβ plaque assembly, through binding to the PAS¹⁴.

According to one aspect of the presently disclosed subject matter, a machine learning approach was employed with literature data followed by a screening strategy for the detection of AChE inhibitors. Then, a docking approach was used to visualize the interactions with the most promising inhibitor identified. Promising machine learning models were generated for eel and human ACHe using literature data along with ASSAY CENTRAL® or several alternative machine learning algorithms. These models were used to score compounds in various commercial libraries. In order to further test and improve these models, the 2558 compounds from the MicroSource library were screened. Initially, several novel inhibitors of eel AChE were identified. One of these was tilorone, an antiviral molecule that is approved for use in several countries. This molecule inhibited eel AChE with an IC₅₀ of 14.4 nM, and human AChE with an average IC₅₀ of 64.6 nM. Several other molecules were identified in the initial study, including agelasine, which has previously been indirectly reported in the extract from the sponge Agelas marmarica, which inhibited acetylcholinesterase, and it can be presumed to also be present in the extract⁵⁸. It should be noted that eel and human AChE machine learning models generated from literature data were able to show greater than random ROC values for the respective test sets (see FIGS. 9A and 9B).

The structure of tilorone has some similarities to tacrine, the first AChE inhibitor approved for the treatment of Alzheimer's Disease, which is a very simple molecule with a three-ring planar structure. See Table 2, below. Due to its hepatotoxicity, tacrine was removed from the market in 1998; however, many groups in the interim have tried to recapitulate the clinical effects of tacrine without the toxicity by creating derivatives based on the three-ring core^{24,59}. While the fluorenone core of tilorone resembles the three planar rings of tacrine, the anti-AChE activity of tilorone does not appear to be reliant on its core structure, as neither fluorenone, nor the close derivative 2,7-dihydroxy-9H-fluoren-9-one inhibit AChE activity. See Table 2. In contrast, two antimalarial molecules quinacrine and pyronaridine that roughly share the three planar rings of tacrine displayed >50% inhibition at 20 μM. This planar three ring core is not a guarantee of activity as the organosulfur lucanthone did not show appreciable activity. Hence, without being bound to any one theory, it appears that the tertiary amines on tilorone can be more important inhibitory features. These are also a common feature shared with tacrine.

TABLE 2
Inhibition of eel AChE activity of selected molecules with tricyclic core structures
at 20 μM.

| | | Percentage |
|---|---|---|
| Structure | Compound | inhibition |
| 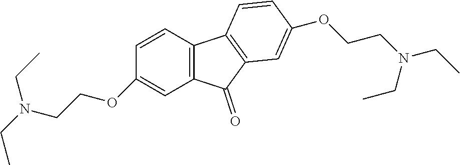 [SMILES: CCN(CC)CCOc1ccc2c(c1)C(=O)c1cc(OCCN(CC)CC)ccc1-2] | tilorone | 102.8 |
| 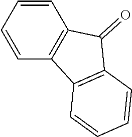 [SMILES: O=C1c2ccccc2-c2ccccc21] | fluorenone | 3 |
| 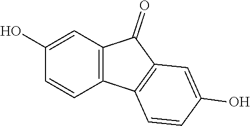 [SMILES: O=C1c2cc(O)ccc2-c2ccc(O)cc21] | 2,7-dihydroxy- 9H-fluoren-9- one | 30 |
| 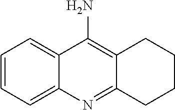 [SMILES: Nc1c2c(nc3ccccc13)CCCC2] | tacrine hydrochloride | 100.6 |
| 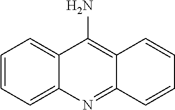 [SMILES: Nc1c2ccccc2nc2ccccc12] | aminacrine | 99.2 |
| 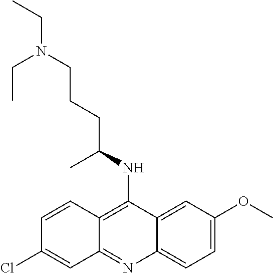 [SMILES: CCN(CC)CCC[C@H](C)Nc1c2ccc(Cl)cc2nc2ccc(OC)cc12] | quinacrine hydrochloride | 61.8 |
| 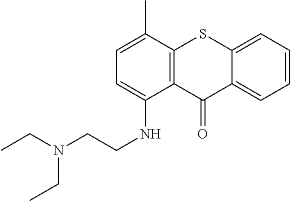 [SMILES: CCN(CC)CCNc1ccc(C)c2sc3ccccc3c(=O)c12] | lucanthone | 11 |
| 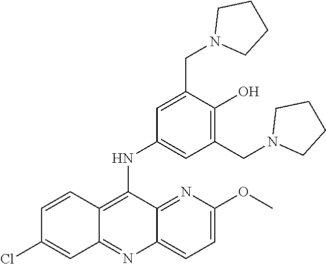 [SMILES: COc1ccc2nc3cc(Cl)ccc3c(Nc3cc(CN4CCCC4)c(O)c(CN4CCCC4)c3)c2n1] | pyronaridine | 53.6 |

Tilorone is sold under the names Lavomax and Amixin and is a small molecule antiviral medication is that is approved for use outside of the United States⁶⁰. Tilorone has also been shown to have anti-cancer properties⁶¹, which have been attributed to the planar ring structure and the ability to intercalate DNA⁶². In recent years, tilorone was discovered to be a potent inhibitor of ebola virus infection in mice, possibly by binding to the viral glycoprotein or through de-acidification of the lysosomal environment required for viral replication⁶³⁻⁶⁵. Tilorone is also an agonist of the α7 nicotinic acetylcholine receptor (α7 nAChR)^{66,67},the most widely expressed nicotinic subunit in the CNS, where most of the nicotinic receptors are found on the pre-synaptic membrane and regulate the release of neurotransmitters like dopamine, glutamate, GABA, norepinephrine, serotonin, and acetylcholine⁶⁸. This would make it of particular interest in AD, where α7 nAChR activation by acetylcholine is hampered by binding of aβ to the receptor and agonists are under investigation as therapeutics^{69,70}.

Some safety concerns associated with tilorone have been reported in the literature. It was reported to be an interferon inducer in mice⁷¹, a quality that could explain some of its potent antiviral properties. It was also reported to induce keratopathy in humans when applied to the cornea, and recent investigations into the lysosomotropic nature of tilorone propose the question of lipidosis^{72,73}. Pharmacological safety profiling of tilorone performed as part of the present studies, however, showed relatively low interaction between tilorone and 43 of the most common targets of drug off-target adverse reactions when tested at 1 μM. See Table 1, above. Interestingly, the target with the next highest interaction with tilorone was a serotonin transporter, where 44% inhibition was observed. While these results were positive with regard to the safety of tilorone, a further approach to overcoming potential safety concerns, e.g., in particular applications or subjects, is the synthesis of tilorone analogs.

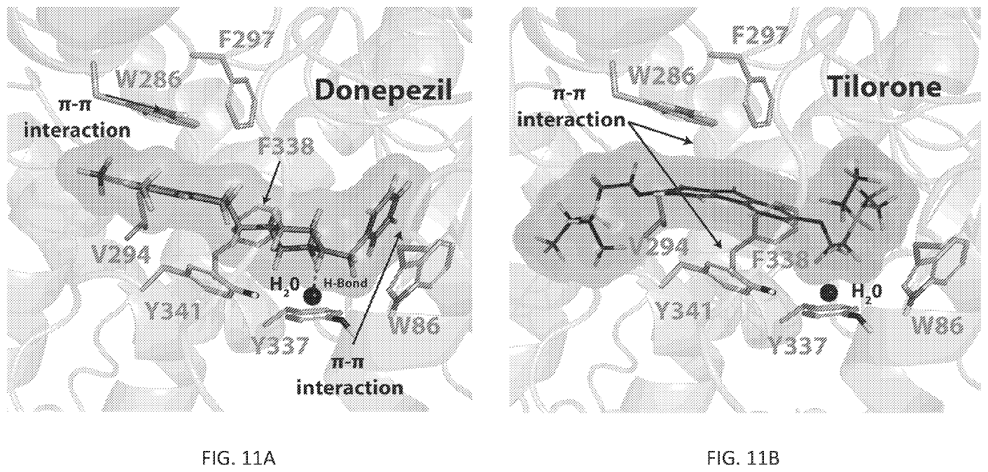

In order to gain insight into how tilorone displays selective inhibition for AChE over BChE, docking experiments with tilorone in AChE were performed. AChE is a fast enzyme, catalyzing the hydrolysis of approximately 25,000 acetylcholine molecules per second, at roughly the speed of diffusion⁷⁴. Without being bound to any one theory, a possible explanation for the speed of AChE catalysis is the binding of acetylcholine first to a peripheral anionic site comprised of five hydrophobic residues at the entrance to a 20 Å deep, narrow gorge leading to the enzyme catalytic site⁷⁵. This gorge itself contains fourteen aromatic residues, and the catalytic site is comprised of two separate binding sites for acetylcholine: the anionic site, which binds the quaternary nitrogen, and the esterase site, where the hydrolysis of acetylcholine takes lace⁷⁴. The catalytic triad of human AChE, consisting of ser203, his447 and glu334 (human AChE numbering), is also present in BChE⁷⁶; however, the active site gorges differ greatly in size. A direct comparison between the human BChE and AChE from Torpedo californicus showed the BChE gorge to be ˜200Å larger than the AChE⁷⁷. The BChE gorge also lacks six of the aromatic residues found in the AChE gorge, which are involved in the specificity of AChE ligand binding^{78,79}. BChE is a closely related enzyme that is most abundant in plasma, and can “scavenge” toxic esters like neurotoxic organophosphates that would otherwise inhibit AChE, as well as larger ligands like cocaine⁸⁰. It is also of interest as a potential drug target in AD therapy because of its increased role in the brain as AChE levels drop⁸¹. Despite this overlapping function, there are enough differences between the enzymes to allow for specific inhibition of each. For example, rivastigmine is a dual inhibitor of AChE and BChE, while galantamine and donepezil are selective AChE inhibitors⁸². While tilorone inhibited human AChE, this activity did not extend to the paralog BChE. Molecular docking of tilorone into human AChE suggests it likely inhibits activity by occupying the active-site gorge in a manner that is similar to donepezil, a cholinesterase inhibitor that is also specific for AChE. In particular, the W86 of the PAS of AChE, that is important for donepezil binding and which seems to exhibit pi-pi interactions with tilorone, is missing in BChE^{76,83}. See FIGS. 11A and 11B.

In conclusion, there are only a few AChE inhibitors that are approved for treatment of AD or other diseases. The discovery of new inhibitors or repurposing of existing drugs can be a route to broadening therapeutic options.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

1. Whitehouse, P. J.; Price, D. L.; Clark, A. W.; Coyle, J. T.; DeLong, M. R., Alzheimer disease: evidence for selective loss of cholinergic neurons in the nucleus basalis. Ann Neurol 1981, 10 (2), 122-6.

2. Gilhus, N. E., Myasthenia Gravis. N Engl. Med 2016, 375 (26), 2570-2581.

3. Jellinger, K. A., Dementia with Lewy bodies and Parkinson's disease-dementia: current concepts and controversies. J Neural Transm (Vienna) 2018, 125 (4), 615-650.

4. Khan, H.; Marya; Amin, S.; Kamal, M. A.; Patel, S., Flavonoids as acetylcholinesterase inhibitors: Current therapeutic standing and future prospects. Biomed Pharmacother 2018, 101, 860-870.

5. Santos, B.; Gonzalez-Fraile, E.; Zabala, A.; Guillen, V.; Rueda, J. R.; Ballesteros,

J., Cognitive improvement of acetylcholinesterase inhibitors in schizophrenia. J Psychopharmacol 2018, 32 (11), 1155-1166.

6. Noufi, P.; Khoury, R.; Jeyakumar, S.; Grossberg, G. T., Use of Cholinesterase Inhibitors in Non-Alzheimer's Dementias. Drugs Aging 2019, 36 (8), 719-731.

7. Dawson, A. H.; Buckley, N. A., Pharmacological management of anticholinergic delirium-theory, evidence and practice. Br J Clin Phannacol 2016, 81 (3), 516-24.

8. Zaki, A. G.; El-Sayed, E. R.; Abd Elkodous, M.; El-Sayyad, G. S., Microbial acetylcholinesterase inhibitors for Alzheimer's therapy: recent trends on extraction, detection, irradiation-assisted production improvement and nano-structured drug delivery. Appl Microbiol Biotechnol 2020, 104 (11), 4717-4735.

9. Li, S.; Liu, C.; Liu, C.; Zhang, Y., Extraction and in vitro screening of potential acetylcholinesterase inhibitors from the leaves of Panax japonicus. J Chromatogr B Analyt Technol Biomed Life Sci 2017, 1061-1062, 139-145.

10. Trinh Thi Thanh, V.; Doan Thi Mai, H.; Pham, V. C.; Litaudon, M.; Dumontet, V.; Gueritte, F.; Nguyen, V. H.; Chau, V. M., Acetylcholinesterase inhibitors from the leaves of Macaranga kurzii. J Nat Prod 2012, 75 (11), 2012-5.

11. Sepcic, K.; Kauferstein, S.; Mebs, D.; Turk, T., Biological activities of aqueous and organic extracts from tropical marine sponges. Mar Drugs 2010, 8 (5), 1550-66.

12. Pandey, S.; Sree, A.; Sethi, D. P.; Kumar, C. G.; Kakollu, S.; Chowdhury, L.; Dash, S. S., A marine sponge associated strain of Bacillus subtilis and other marine bacteria can produce anticholinesterase compounds. Microb Cell Fact 2014, 13 (1), 24.

13. Wiemann, J.; Karasch, J.; Loesche, A.; Heller, L.; Brandt, W.; Csuk, R., Piperlongumine B and analogs are promising and selective inhibitors for acetylcholinesterase. Eur J Med Chem 2017, 139, 222-231.

14. Wu, Z.; Liao, W.; Chen, K.; Qin, J.; Tang, H., Synthesis of derivatives of cleistopholine and their anti-acetylcholinesterase and anti-beta-amyloid aggregation activity. Bioorg Chem 2018, 76, 228-236.

15. de Lima, B. R.; Lima, J. M.; Maciel, J. B.; Valentim, C. Q.; Nunomura, R. C. S.; Lima, E. S.; Koolen, H. H. F.; de Souza, A. D. L.; Pinheiro, M. L. B.; Cass, Q. B.; da Silva, F. M. A., Synthesis and Inhibition Evaluation of New Benzyltetrahydroprotoberberine Alkaloids Designed as Acetylcholinesterase Inhibitors. Front Chem 2019, 7, 629.

16. Wiemann, J.; Loesche, A.; Csuk, R., Novel dehydroabietylamine derivatives as potent inhibitors of acetylcholinesterase. Bioorg Chem 2017, 74, 145-157.

17. Uriarte-Pueyo, I.; Calvo, M. I., Flavonoids as acetylcholinesterase inhibitors. Curr Med Chem 2011, 18 (34), 5289-302.

18. Mughal, E. U.; Sadiq, A.; Ashraf, J.; Zafar, M. N.; Sumrra, S. H.; Tariq, R.; Mumtaz, A.; Javid, A.; Khan, B. A.; Ali, A.; Javed, C. O., Flavonols and 4-thioflavonols as potential acetylcholinesterase and butyrylcholinesterase inhibitors: Synthesis, structure-activity relationship and molecular docking studies. Bioorg Chem 2019, 91, 103124.

19. Anand, P.; Singh, B.; Singh, N., A review on coumarins as acetylcholinesterase inhibitors for Alzheimer's disease. Bioorg Med Chem 2012, 20 (3), 1175-80.

20. de Souza, G. A.; da Silva, S. J.; Del Cistia, C. N.; Pitasse-Santos, P.; Pires, L. O.; Passos, Y. M.; Cordeiro, Y.; Cardoso, C. M.; Castro, R. N.; Sant'Anna, C. M. R.; Kummerle, A. E., Discovery of novel dual-active 3-(4-(dimethylamino)phenyl)-7-aminoalcoxy-coumarin as potent and selective acetylcholinesterase inhibitor and antioxidant. J Enzyme Inhib Med Chem 2019, 34 (1), 631-637.

21. Sahin, Z.; Ertas, M.; Bender, C.; Bulbul, E. F.; Berk, B.; Biltekin, S. N.; Yurttas, L.; Demirayak, S., Thiazole-substituted benzoylpiperazine derivatives as acetylcholinesterase inhibitors. Drug Dev Res 2018, 79 (8), 406-425.

22. Zhou, Y.; Sun, W.; Peng, J.; Yan, H.; Zhang, L.; Liu, X.; Zuo, Z., Design, synthesis and biological evaluation of novel copper-chelating acetylcholinesterase inhibitors with pyridine and N-benzylpiperidine fragments. Bioorg Chem 2019, 93, 103322.

23. Stavrakov, G.; Philipova, I.; Zheleva-Dimitrova, D.; Valkova, I.; Salamanova, E.; Konstantinov, S.; Doytchinova, I., Docking-based design and synthesis of galantamine-camphane hybrids as inhibitors of acetylcholinesterase. Chem Biol Drug Des 2017, 90 (5), 709-718.

24. Tanoli, S. T.; Ramzan, M.; Hassan, A.; Sadiq, A.; Jan, M. S.; Khan, F. A.; Ullah, F.; Ahmad, H.; Bibi, M.; Mahmood, T.; Rashid, U., Design, synthesis and bioevaluation of tricyclic fused ring system as dual binding site acetylcholinesterase inhibitors. Bioorg Chem 2019, 83, 336-347.

25. Makhaeva, G. F.; Kovaleva, N. V.; Lushchekina, S. V.; Rudakova, E. V.; Boltneva, N. P.; Proshin, A. N.; Lednev, B. V.; Serkov, I. V.; Bachurin, S. O., Conjugates of Tacrine and Its Cyclic Homologues with p-Toluenesulfonamide as Novel Acetylcholinesterase and Butyrylcholinesterase Inhibitors. Dokl Biochem Biophys 2018, 483 (1), 369-373.

26. Skibinski, R.; Czarnecka, K.; Girek, M.; Bilichowski, I.; Chufarova, N.; Mikiciuk-Olasik, E.; Szymanski, P., Novel tetrahydroacridine derivatives with iodobenzoic acid moiety as multifunctional acetylcholinesterase inhibitors. Chem Biol Drug Des 2018, 91 (2), 505-518.

27. Bolognesi, M. L.; Bartolini, M.; Mancini, F.; Chiriano, G.; Ceccarini, L.; Rosini, M.; Milelli, A.; Tumiatti, V.; Andrisano, V.; Melchiorre, C., Bis(7)-tacrine derivatives as multitarget-directed ligands: Focus on anticholinesterase and antiamyloid activities. ChemMedChem 2010, 5 (8), 1215-20.

28. Pandolfi, F.; De Vita, D.; Bortolami, M.; Coluccia, A.; Di Santo, R.; Costi, R.; Andrisano, V.; Alabiso, F.; Bergamini, C.; Fato, R.; Bartolini, M.; Scipione, L., New pyridine derivatives as inhibitors of acetylcholinesterase and amyloid aggregation. Eur J Med Chem 2017, 141, 197-210.

29. De Vita, D.; Pandolfi, F.; Ornano, L.; Feroci, M.; Chiarotto, I.; Sileno, I.; Pepi, F.; Costi, R.; Di Santo, R.; Scipione, L., New N,N-dimethylcarbamate inhibitors of acetylcholinesterase: design synthesis and biological evaluation. J Enzyme Inhib Med Chem 2016, 31 (sup4), 106-113.

30. Shiri, F.; Pirhadi, S.; Ghasemi, J. B., Dynamic structure based pharmacophore modeling of the Acetylcholinesterase reveals several potential inhibitors. J Biomol Struct Dyn 2019, 37 (7), 1800-1812.

31. Chekmarev, D.; Kholodovych, V.; Kortagere, S.; Welsh, W. J.; Ekins, S., Predicting inhibitors of acetylcholinesterase by regression and classification machine learning approaches with combinations of molecular descriptors. Pharm Res 2009, 26 (9), 2216-24.

32. Zhang, L.; Li, D.; Cao, F.; Xiao, W.; Zhao, L.; Ding, G.; Wang, Z. Z., Identification of Human Acetylcholinesterase Inhibitors from the Constituents of EGb761 by Modeling Docking and Molecular Dynamics Simulations. Comb Chem High Throughput Screen 2018, 21 (1), 41-49.

33. Iman, K.; Mirza, M. U.; Mazhar, N.; Vanmeert, M.; Irshad, I.; Kamal, M. A., In silico Structure-based Identification of Novel Acetylcholinesterase Inhibitors Against Alzheimer's Disease. CNS Neurol Disord Drug Targets 2018, 17 (1), 54-68.

34. Telpoukhovskaia, M. A.; Patrick, B. O.; Rodriguez-Rodriguez, C.; Orvig, C., In silico to in vitro screening of hydroxypyridinones as acetylcholinesterase inhibitors. Bioorg Med Chem Lett 2016, 26 (6), 1624-1628.

35. Cui, L.; Wang, Y.; Liu, Z.; Chen, H.; Wang, H.; Zhou, X.; Xu, J., Discovering New Acetylcholinesterase Inhibitors by Mining the Buzhongyiqi Decoction Recipe Data. J Chem Inf Model 2015, 55 (11), 2455-63.

36. Muthukumaran, P.; Rajiniraja, M., MIA-QSAR based model for bioactivity prediction of flavonoid derivatives as acetylcholinesterase inhibitors. J Theor Biol 2018, 459, 103-110.

37. Jang, C.; Yadav, D. K.; Subedi, L.; Venkatesan, R.; Venkanna, A.; Afzal, S.; Lee, E.; Yoo, J.; Ji, E.; Kim, S. Y.; Kim, M. H., Identification of novel acetylcholinesterase inhibitors designed by pharmacophore-based virtual screening, molecular docking and bioassay. Sci Rep 2018, 8 (1), 14921.

38. de Almeida, J. R.; Figueiro, M.; Almeida, W. P.; de Paula da Silva, C. H. T., Discovery of novel dual acetylcholinesterase inhibitors with antifibrillogenic activity related to Alzheimer's disease. Future Med Chem 2018, 10 (9), 1037-1053.

39. Jiang, C. S.; Ge, Y. X.; Cheng, Z. Q.; Song, J. L.; Wang, Y. Y.; Zhu, K.; Zhang, H., Discovery of new multifunctional selective acetylcholinesterase inhibitors: structure-based virtual screening and biological evaluation. J Comput Aided Mol Des 2019, 33 (5), 521-530.

40. Anantpadma, M.; Lane, T.; Zorn, K. M.; Lingerfelt, M. A.; Clark, A. M.; Freundlich, J. S.; Davey, R. A.; Madrid, P. B.; Ekins, S., Ebola Virus Bayesian Machine Learning Models Enable New in Vitro Leads. ACS Omega 2019, 4 (1), 2353-2361.

41. Dalecki, A. G.; Zorn, K. M.; Clark, A. M.; Ekins, S.; Narmore, W. T.; Tower, N.; Rasmussen, L.; Bostwick, R.; Kutsch, O.; Wolschendorf, F., High-throughput screening and Bayesian machine learning for copper-dependent inhibitors of Staphylococcus aureus. Metallomics 2019, 11 (3), 696-706.

42. Ekins, S.; Gerlach, J.; Zorn, K. M.; Antonio, B. M.; Lin, Z.; Gerlach, A., Repurposing Approved Drugs as Inhibitors of Kv7.1 and Nav1.8 to Treat Pitt Hopkins Syndrome. Pharm Res 2019, 36 (9), 137.

43. Ekins, S.; Puhl, A. C.; Zorn, K. M.; Lane, T. R.; Russo, D. P.; Klein, J. J.; Hickey, A. J.; Clark, A. M., Exploiting machine learning for end-to-end drug discovery and development. Nat Mater 2019, 18 (5), 435-441.

44. Hernandez, H. W.; Soeung, M.; Zorn, K. M.; Ashoura, N.; Mottin, M.; Andrade, C. H.; Caffrey, C. R.; de Siqueira-Neto, J. L.; Ekins, S., High Throughput and Computational Repurposing for Neglected Diseases. Pharm Res 2018, 36 (2), 27.

45. Lane, T.; Russo, D. P.; Zorn, K. M.; Clark, A. M.; Korotcov, A.; Tkachenko, V.; Reynolds, R. C.; Perryman, A. L.; Freundlich, J. S.; Ekins, S., Comparing and Validating Machine Learning Models for Mycobacterium tuberculosis Drug Discovery. Mol Pharm 2018, 15 (10), 4346-4360.

46. Russo, D. P.; Zorn, K. M.; Clark, A. M.; Zhu, H.; Ekins, S., Comparing Multiple Machine Learning Algorithms and Metrics for Estrogen Receptor Binding Prediction. Mol Pharm 2018, 15 (10), 4361-4370.

47. Sandoval, P. J.; Zorn, K. M.; Clark, A. M.; Ekins, S.; Wright, S. H., Assessment of Substrate-Dependent Ligand Interactions at the Organic Cation Transporter OCT2 Using Six Model Substrates. Mol Pharmacol 2018, 94 (3), 1057-1068.

48. Wang, P. F.; Neiner, A.; Lane, T. R.; Zorn, K. M.; Ekins, S.; Kharasch, E. D., Halogen Substitution Influences Ketamine Metabolism by Cytochrome P450 2B6: In Vitro and Computational Approaches. Mol Pharm 2019, 16 (2), 898-906.

49. Zorn, K. M.; Lane, T. R.; Russo, D. P.; Clark, A. M.; Makarov, V.; Ekins, S., Multiple Machine Learning Comparisons of HIV Cell-based and Reverse Transcriptase Data Sets. Mol Pharm 2019, 16 (4), 1620-1632.

50. Willighagen, E. L.; Mayfield, J. W.; Alvarsson, J.; Berg, A.; Carlsson, L.; Jeliazkova, N.; Kuhn, S.; Pluskal, T.; Rojas-Cherto, M.; Spjuth, O.; Torrance, G.; Evelo, C. T.; Guha, R.; Steinbeck, C., The Chemistry Development Kit (CDK) v2.0: atom typing, depiction, molecular formulas, and substructure searching. J Cheminfonn 2017, 9 (1), 33.

51. Clark, A. M.; Dole, K.; Coulon-Spektor, A.; McNutt, A.; Grass, G.; Freundlich, J. S.; Reynolds, R. C.; Ekins, S., Open Source Bayesian Models. 1. Application to ADME/Tox and Drug Discovery Datasets. J Chem Inf Model 2015, 55 (6), 1231-45.

52. Ekins, S.; Mottin, M.; Ramos, P. R. P. S.; Sousa, B. K. P.; Neves, B. J.; Foil, D. H.; Zorn, K. M.; Braga, R. C.; Coffee, M.; Southan, C.; Puhl, A. C.; Andrade, C. H., Déjà. vu: Stimulating Open Drug Discovery for SARS-CoV-2. Drug Discov Today 2020, In press.

53. Ellman, G. L.; Courtney, K. D.; Andres, V., Jr.; Feather-Stone, R. M., A new and rapid colorimetric determination of acetylcholinesterase activity. Biochem Pharmacol 1961, 7, 88-95.

54. Wu, G.; Robertson, D. H.; Brooks, C. L., 3rd; Vieth, M., Detailed analysis of grid-based molecular docking: A case study of CDOCKER-A CHARMm-based MD docking algorithm. J Comput Chem 2003, 24 (13), 1549-62.

55. Jbilo, O.; L'Hermite, Y.; Talesa, V.; Toutant, J. P.; Chatonnet, A., Acetylcholinesterase and butyrylcholinesterase expression in adult rabbit tissues and during development. Eur Biochem 1994, 225 (1), 115-24.

56. Giacobini, E., Selective inhibitors of butyrylcholinesterase: a valid alternative for therapy of Alzheimer's disease? Drugs Aging 2001, 18 (12), 891-8.

57. Wessler, I.; Kirkpatrick, C. J., Acetylcholine beyond neurons: the non-neuronal cholinergic system in humans. Br J Pharmacol 2008, 154 (8), 1558-71.

58. Beedessee, G.; Ramanjooloo, A.; Surnam-Boodhun, R.; van Soest, R. W.; Marie, D. E., Acetylcholinesterase-inhibitory activities of the extracts from sponges collected in mauritius waters. Chem Biodivers 2013, 10 (3), 442-51.

59. Uldall, P.; Hansen, F. J.; Tonnby, B., Lamotrigine in Rett syndrome. Neuropediatrics 1993, 24 (6), 339-40.

60. Ekins, S.; Lingerfelt, M. A.; Comer, J. E.; Freiberg, A. N.; Mirsalis, J. C.; O'Loughlin, K.; Harutyunyan, A.; McFarlane, C.; Green, C. E.; Madrid, P. B., Efficacy of Tilorone Dihydrochloride against Ebola Virus Infection. Antimicrob Agents Chemother 2018, 62 (2).

61. Morahan, P. S.; Munson, J. A.; Baird, L. G.; Kaplan, A. M.; Regelson, W., Antitumor action of pyran copolymer and tilorone against Lewis lung carcinoma and B-16 melanoma. Cancer Res 1974, 34 (3), 506-11.

62. Zhou, D.; Tuo, W.; Hu, H.; Xu, J.; Chen, H.; Rao, Z.; Xiao, Y.; Hu, X.; Liu, P., Synthesis and activity evaluation of tilorone analogs as potential anticancer agents. Eur J Med Chem 2013, 64, 432-41.

63. Lane, T. R.; Dyall, J.; Mercer, L.; Goodin, C.; Foil, D. H.; Zhou, H.; Postnikova, E.; Liang, J. Y.; Holbrook, M. R.; Madrid, P. B.; Ekins, S. Repurposing Pyramax® for the Treatment of Ebola Virus Disease: Additivity of the Lysosomotropic Pyronaridine and Non-Lysosomotropic Artesunate; DOI: 10.1101/2020.04.25.061333v1.

64. Lane, T. R.; Ekins, S., Towards the Target: Tilorone, Quinacrine and Pyronaridine Bind to Ebola Virus Glycoprotein. ACS Med Chem Lett 2020, 2020.05.26.118182.

65. Lane, T. R.; Massey, C.; Comer, J. E.; Anantpadma, M.; Freundlich, J. S.; Davey, R. A.; Madrid, P. B.; Ekins, S., Repurposing the antimalarial pyronaridine tetraphosphate to protect against Ebola virus infection. PLoS Negl Trop Dis 2019, 13 (11), e0007890.

66. Schrimpf, M. R.; Sippy, K. B.; Briggs, C. A.; Anderson, D. J.; Li, T.; Ji, J.; Frost, J. M.; Surowy, C. S.; Bunnelle, W. H.; Gopalakrishnan, M.; Meyer, M. D., SAR of alpha7 nicotinic receptor agonists derived from tilorone: exploration of a novel nicotinic pharmacophore. Bioorg Med Chem Lett 2012, 22 (4), 1633-8.

67. Briggs, C. A.; Schrimpf, M. R.; Anderson, D. J.; Gubbins, E. J.; Gronlien, J. H.; Hakerud, M.; Ween, H.; Thorin-Hagene, K.; Malysz, J.; Li, J.; Bunnelle, W. H.; Gopalakrishnan, M.; Meyer, M. D., alpha7 nicotinic acetylcholine receptor agonist properties of tilorone and related tricyclic analogues. Br J Pharmacol 2008, 153 (5), 1054-61.

68. Ferreira-Vieira, T. H.; Guimaraes, I. M.; Silva, F. R.; Ribeiro, F. M., Alzheimer's disease: Targeting the Cholinergic System. Curr Neuropharmacol 2016, 14 (1), 101-15.

69. Fabiani, C.; Antollini, S. S., Alzheimer's Disease as a Membrane Disorder: Spatial Cross-Talk Among Beta-Amyloid Peptides, Nicotinic Acetylcholine Receptors and Lipid Rafts. Front Cell Neurosci 2019, 13, 309.

70. Yang, T.; Xiao, T.; Sun, Q.; Wang, K., The current agonists and positive allosteric modulators of alpha7 nAChR for CNS indications in clinical trials. Acta Pharm Sin B 2017, 7 (6), 611-622.

71. Stringfellow, D. A.; Glasgow, L. A., Tilorone hydrochloride: an oral interferon-inducing agent. Antimicrob Agents Chemother 1972, 2 (2), 73-8.

72. Fischer, J.; Hein, L.; Lullmann-Rauch, R.; von Witzendorff, B., Tilorone-induced lysosomal lesions: the bisbasic character of the drug is essential for its high potency to cause storage of sulphated glycosaminoglycans. Biochem J 1996, 315 (Pt 2), 369-75.

73. Fischer, J., Tilorone-induced lysosomal storage of glycosaminoglycans in cultured corneal fibroblasts: biochemical and physicochemical investigations. Biochem J 1995, 312 (Pt 1), 215-22.

74. Colovic, M. B.; Krstic, D. Z.; Lazarevic-Pasti, T. D.; Bondzic, A. M.; Vasic, V. M., Acetylcholinesterase inhibitors: pharmacology and toxicology. Curr Neurophannacol 2013, 11 (3), 315-35.

75. Sussman, J. L.; Harel, M.; Frolow, F.; Oefner, C.; Goldman, A.; Toker, L.; Silman, I., Atomic structure of acetylcholinesterase from Torpedo californica: a prototypic acetylcholine-binding protein. Science 1991, 253 (5022), 872-9.

76. Macdonald, I. R.; Martin, E.; Rosenberry, T. L.; Darvesh, S., Probing the peripheral site of human butyrylcholinesterase. Biochemistry 2012, 51 (36), 7046-53.

77. Saxena, A.; Redman, A. M.; Jiang, X.; Lockridge, O.; Doctor, B. P., Differences in active-site gorge dimensions of cholinesterases revealed by binding of inhibitors to human butyrylcholinesterase. Chem Biol Interact 1999, 119-120, 61-9.

78. Harel, M.; Sussman, J. L.; Krejci, E.; Bon, S.; Chanal, P.; Massoulie, J.; Silman, I., Conversion of acetylcholinesterase to butyrylcholinesterase: modeling and mutagenesis. Proc Natl Acad Sci USA 1992, 89 (22), 10827-31.

79. Radic, Z.; Pickering, N. A.; Vellom, D. C.; Camp, S.; Taylor, P., Three distinct domains in the cholinesterase molecule confer selectivity for acetyl- and butyrylcholinesterase inhibitors. Biochemistry 1993, 32 (45), 12074-84.

80. Nicolet, Y.; Lockridge, O.; Masson, P.; Fontecilla-Camps, J. C.; Nachon, F., Crystal structure of human butyrylcholinesterase and of its complexes with substrate and products. J Biol Chem 2003, 278 (42), 41141-7.

81. Nordberg, A.; Ballard, C.; Bullock, R.; Darreh-Shori, T.; Somogyi, M., A review of butyrylcholinesterase as a therapeutic target in the treatment of Alzheimer's disease. Prim Care Companion CNS Disord 2013, 15 (2).

82. Sharma, K., Cholinesterase inhibitors as Alzheimer's therapeutics (Review). Mol Med Rep 2019, 20 (2), 1479-1487.

83. Cheung, J.; Rudolph, M. J.; Burshteyn, F.; Cassidy, M. S.; Gary, E. N.; Love, J.; Franklin, M. C.; Height, J. J., Structures of human acetylcholinesterase in complex with pharmacologically important ligands. J Med Chem 2012, 55 (22), 10282-6.

84. Chapleau R R, Robinson P J, Schlager J J, Gearhart J M. Potential new therapeutic modality revealed through agent-based modeling of the neuromuscular junction and acetylcholinesterase inhibition. Theoretical biology & medical modelling. 2014;11:42.

85. Chapleau R R, McElroy C A, Ruark C D, Fleming E J, Ghering A B, Schlager J J, Poeppelman L D, Gearhart J M. High-Throughput Screening for Positive Allosteric Modulators Identified Potential Therapeutics against Acetylcholinesterase Inhibition. Journal of biomolecular screening. 2015; 20(9):1142-9.

86. Radić Z, Taylor P. The influence of peripheral site ligands on the reaction of symmetric and chiral organophosphates with wildtype and mutant acetylcholinesterases. Chem Biol Interact. 1999; 119-120:111-7.

87. Radić Z, Taylor P. Peripheral site ligands accelerate inhibition of acetylcholinesterase by neutral organophosphates. J Appl Toxicol. 2001;21 Suppl 1:S13-4.

88. Harris L W, Heyl W C, Stitcher D L, Broomfield C A. Effects of 1,1′-oxydimethylene bis-(4-tert-butylpyridinium chloride) (SAD-128) and decamethonium on reactivation of soman- and sarin-inhibited cholinesterase by oximes. Biochem Pharmacol. 1978; 27(5):757-61.

89. Luo C, Ashani Y, Doctor B P. Acceleration of oxime-induced reactivation of organophosphate-inhibited fetal bovine serum acetylcholinesterase by monoquaternary and bisquaternary ligands. Mol Pharmacol. 1998; 53(4):718-26.

90. Schoene K. Aging of soman-inhibited acetylcholinesterase: inhibitors and accelerators. Biochim Biophys Acta. 1978; 525(2):468-71.

91. Lilienfeld S. Galantamine—a novel cholinergic drug with a unique dual mode of action for the treatment of patients with Alzheimer's disease. CNS drug reviews. 2002; 8(2):159-76.

92. Albuquerque E X, Pereira E F, Aracava Y, Fawcett W P, Oliveira M, Randall W R, Hamilton T A, Kan R K, Romano J A, Jr., Adler M. Effective countermeasure against poisoning by organophosphorus insecticides and nerve agents. Proc Natl Acad Sci U S A. 2006; 103(35):13220-5.

93. Hilmas C J, Poole M J, Finneran K, Clark M G, Williams P T. Galantamine is a novel post-exposure therapeutic against lethal VX challenge. Toxicology and applied pharmacology. 2009; 240(2):166-73.

94. Lane M, Carter D, Pescrille J D, Aracava Y, Fawcett W P, Basinger G W, Pereira E F R, Albuquerque E X. Oral Pretreatment with Galantamine Effectively Mitigates the Acute Toxicity of a Supralethal Dose of Soman in Cynomolgus Monkeys Posttreated with Conventional Antidotes. J Pharmacol Exp Ther. 2020; 375(1):115-26.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of inhibiting acetylcholinesterase (AChE) in a sample comprising AChE, wherein the method comprises contacting the AChE in the sample with an effective amount of at least one compound selected from the group consisting of tilorone, a tilorone analog, cetylpyridinium, bezedoxifene acetate, rifaximin, dequalinium chloride, agelasine, and pharmaceutically acceptable salts thereof.

2. The method of claim 1, wherein the at least one compound is selected from tilorone, a tilorone analog and/or a pharmaceutically acceptable salt thereof, wherein said tilorone analog is a selective AChE inhibitor.

3. The method of claim 1 or claim 2, wherein the at least one compound has a 50% inhibitory concentration (IC₅₀) for human and/or eel AChE of about 100 nanomolar (nM) or less.

4. The method of claim 3, wherein the at least one compound has an IC₅₀ for human AChE of about 75 nM or less and/or an IC₅₀ for eel AChE of about 15 nM or less.

5. The method of any one of claims 1-4, wherein the at least one compound has an IC₅₀ for human or eel AChE that is at least about 100 times lower than an IC₅₀ of the at least one compound for butyrylcholinesterase (BuChE), optionally wherein the at least one compound has an IC₅₀ for human or eel AChE that is at least about 1000 times lower than an IC₅₀ of the at least one compound for BuChE.

6. The method of any one of claims 1-5, wherein the tilorone or tilorone analog and/or pharmaceutically acceptable salt thereof exhibits pi-pi interactions with tryptophan 286 (W286) of the peripheral anionic site (PAS) of human AChE.

7. The method of any one of claims 1-6, wherein the sample comprises a biological fluid, cell, cell extract, tissue, tissue extract, organ or whole organism.

8. The method of any one of claims 1-7, wherein the tilorone or tilorone analog and/or pharmaceutically acceptable salt thereof has a structure of one of Formula (I) and Formula (II): 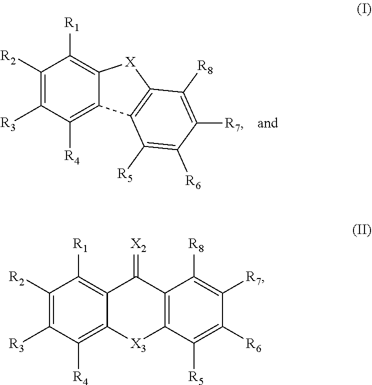
wherein: [symbol]
is absent or a single bond;
X is selected from the group consisting of —C(═Z)—, —S(═O)₂—, —CH₂—, —O—, —S—, and —NH—;
Z is selected from O, S, and CH₂;
X₂ is selected from O, S, and CH₂;
X₃ is selected from —C(═Z)—, —CH₂—, —O—, —S—, and —NH—;
each of R₁, R₂, R₃, R₄, R₅, R₆, R₇, and R₈ is independently selected from the group consisting of H, alkyl, amino, hydroxy, alkoxy, and —X₄-L-N(R₉)₂;
X₄ is selected from —O—, —S—, —NH—C(═O)—, —O—C(═O)—, —C(═O)—, —C(OH)—, and —C(═O)—O—;
L is a bivalent linker moiety, optionally a C₁-C₆ alkylene group; and
each R₉ is alkyl, optionally C₁-C₆ alkyl; aralkyl, or aryl, or wherein two R₉ together form a cyclic bivalent group; or
a pharmaceutically acceptable salt thereof.

9. The method of claim 8, wherein at least one of R₁-R₈ is —X₄-L-N(R₉)₂, optionally wherein one of R₁-R₄ is —X₄-L-N(R₉)₂ and one of R₅-R₈ is X₄-L-N(R₉)₂.

10. A method of treating or preventing a disease, disorder, or condition treatable or preventable by inhibition of acetylcholinesterase (AChE) in a subject in need of treatment thereof and/or of extending the lifespan of a subject, wherein the disease, disorder or condition treatable or preventable by inhibition of AChE is selected from the group consisting of a dermatological disorder, myasthenia gravis, glaucoma, multiple sclerosis, autoimmune encephalomyelitis, organophosphorous (OP) poisoning, nerve agent poisoning, and anticholinergic poisoning, wherein the method comprises administering to the subject an effective amount of at least one compound selected from the group consisting of tilorone, a tilorone analog, cetylpyridinium, dequalinium chloride, bezedoxifene acetate, rifaximin, agelasine, and pharmaceutically acceptable salts thereof.

11. The method of claim 10, wherein the method comprises administering to the subject an effective amount of tilorone or an analog and/or pharmaceutically acceptable salt thereof, wherein said tilorone analog is a selective inhibitor of AChE.

12. The method of claim 10 or claim 11, wherein the subject is a subject suffering from or suspected to be suffering from OP or nerve agent poisoning or is at risk of OP or nerve agent poisoning.

13. The method of claim 12, wherein the subject is at risk for OP or nerve agent poisoning, and the subject is administered the at least one compound prior a potential exposure to an OP or a nerve agent.

14. The method of claim 12 or claim 13, further comprising administering to said subject one or more additional treatment agents for OP or nerve agent poisoning, optionally wherein said one or more additional treatment agents are selected from the group consisting of atropine, pralidoxime or another oxime, a benzodiazepine, and physostigmine salicylate.

15. The method of claim 10 or claim 11, wherein the disease, disorder, or condition treatable or preventable by inhibition of AChE is a dermatological disorder selected from the group consisting of a condition associated with Domodex brevis and/or Demodex folliculorum mites, a bacterial infection, acne, seborrheic dermatitis, perioral dermatitis, acneform rash, transient acantholytic dermatosis, acne necrotica milliaris, steroid induced dermatitis, primary irritation dermatitis, and rosacea.

16. The method of any one of claims 10-15, wherein the at least one compound has a 50% inhibitory concentration (IC₅₀) for eel and/or human AChE of about 100 nanomolar (nM) or less.

17. The method of claim 16, wherein the at least one compound has an IC₅₀ for human AChE of about 75 nM or less and/or an IC₅₀ for eel AChE of about 15 nM or less.

18. The method of any one of claims 10-17, wherein the at least one compound has an IC₅₀ for human or eel AChE that is at least about 100 times lower than an IC₅₀ of the at least one compound for butyrylcholinesterase (BuChE), optionally wherein the at least one compound has an IC₅₀ for human or eel AChE that is at least about 1000 times lower than an IC₅₀ of the at least one compound for BuChE.

19. The method of any one of claims 10-18, wherein the subject is a mammal, optionally a human.

20. The method of any one of claims 10-19, wherein the administering is performed via one of the group consisting of oral administration, intravenous (IV) administration, intraperitoneal (IP) administration, topical administration, intracerebroventricular (ICV) administration, and intrathecal (IT) administration.

21. The method of any one of claims 10-20, wherein the tilorone, tilorone analog and/or pharmaceutically acceptable salt thereof has a structure of one of Formula (I) and Formula (II): 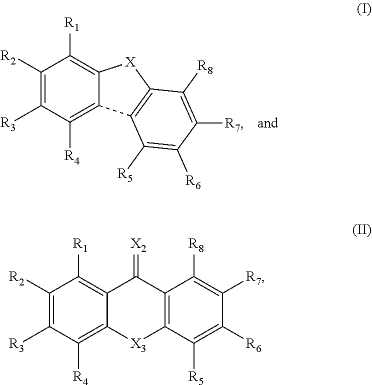
wherein: [symbol]
is absent or a single bond;
X is selected from the group consisting of —C(═Z)—, —S(═O)₂—, —CH₂—, —O—, —S—, and —NH—;
Z is selected from O, S, and CH₂;
X₂ is selected from O, S, and CH₂;
X₃ is selected from —C(═Z)—, —CH₂—, —O—, —S—, and —NH—;
each of R₁, R₂, R₃, R₄, R₅, R₆, R₇, and R₈ is independently selected from the group consisting of H, alkyl, amino, hydroxy, alkoxy, and —X₄-L-N(R₉)₂;
X₄ is selected from —O—, —S—, —NH—C(═O)—, —O—C(═O)—, —C(═O)—, —C(OH)—, and —C(═O)—O—;
L is a bivalent linker moiety, optionally a C₁-C₆ alkylene group; and
each R₉ is alkyl, optionally C₁-C₆ alkyl; aralkyl, or aryl, or wherein two R₉ together form a cyclic bivalent group; or
a pharmaceutically acceptable salt thereof.

22. The method of claim 21, wherein at least one of R₁-R₈ is —X₄-L-N(R₉)₂, optionally wherein one of R₁-R₄ is —X₄-L-N(R₉)₂ and one of R₅-R₈ is X₄-L-N(R₉)₂.

23. The method of any one of claims 10-22, further comprising administering to the subject one or more additional therapeutic agents.
